(19) United States
(12) Patent Application Publication
(10) Pub. No.: US 2025/0275935 A1
(43) Pub. Date: Sep. 4, 2025

(54) COMPOSITIONS AND METHODS FOR TREATING PATIENTS WITH DEMENTIA DUE TO ALZHEIMER'S DISEASE WITH A COMBINATION OF THC AND MELATONIN

(71) Applicant: IGC Pharma IP, LLC, Potomac, MD (US)

(72) Inventors: Ramachandra MUKUNDA, Potomac, MD (US); Jagadeesh S. RAO, Ashburn, VA (US)

(73) Assignee: IGC Pharma IP, LLC, Potomac, MD (US)

(21) Appl. No.: 18/692,705

(22) PCT Filed: Sep. 16, 2022
(86) PCT No.: PCT/US2022/076579

§ 371 Date: Mar. 15, 2024

Related U.S. Application Data
(60) Provisional application No. 63/245,799, filed on Sep. 17, 2021.

(51) Int. Cl. A61K 31/352; A61K 31/12; A61K 31/4045; A61K 31/7048; A61K 36/9066; A61K 47/26; A61K 47/34

(52) U.S. Cl. CPC A61K 31/352; A61K 31/12; A61K 31/4045; A61K 31/7048; A61K 36/9066; A61K 47/26; A61K 47/34

(57) ABSTRACT

Compositions and products (e.g., kits, medical devices, medicaments), which incorporate the compositions, comprising a tetrahydrocannabinol (THC) compound and melatonin compound are provided, Methods for treatment using the compositions and products, methods for diagnosis before or after treatment, and processes for formulating the compositions or manufacturing the products are also provided. They address the need for new treatments of cognitive and perceptual deficits, neuropsychiatric symptoms, neurodegenerative diseases, and neurologic disorders. The inventors discovered therapeutically effective amounts of TI-IC and melatonin can be administered to treat patients, without need for an entourage effect or requirement of cannabidiol (CBD) and other cannabinoid compounds. Optionally, polyphenolic compounds (e.g., curcumin-related compounds, rutin-related compounds) and/or Vitamin E-related compounds (e.g., mixed tocopherols, alpha-tocopherol, beta-tocopherol, gamma-tocopherol) may be administered together with or separately from TI-IC and melatonin compounds.

### TECHNICAL FIELD

The invention relates to compositions, processes for formulating the compositions, methods for treatment and diagnosis, products (e.g., kits, medical devices, medicaments), and processes for manufacturing the products. They address the need for treating cognitive and perceptual deficits, neuropsychiatric symptoms, neurodegenerative diseases, and neurologic disorders.

### BACKGROUND

Dementia describes a group of diseases characterized by serious cognitive decline inter-fering with physical activities of daily living, which are basic and essential tasks routinely performed by healthy adults who do not have a physical or developmental disability. The most common (60%-80%) type of dementia is Alzheimer's disease (AD). Less common are the dementia of other neurodegenerative diseases (e.g., Parkinson's disease), vascular dementia, Lewy body dementia, frontotemporal dementia, and mixed dementia. As implied by its name, the cognitive decline of mild cognitive impairment (MCI) is more than that expected for normal aging but less severe than that of dementia. Symptoms of MCI may remain stable for years, or even improve. In contrast, the cognitive decline of AD is progressive and irreversible. Whether MCI represents a transitional stage in development of dementia and is a significant risk factor for dementia are uncertain.

Although the prevalence of AD increases dramatically in the elderly and the most significant risk factor for late-onset AD is late age, dementia and AD are not inevitable conse-quences of normal aging. Hallmarks within the brain of AD patients include the following: deposition of insoluble amyloid in plaques between cells and the walls of cerebral arteries, hyperphosphorylation of tau protein and their aggregation into insoluble neurofibrillary tangles, loss of neurons and neuronal connectivity, breakdown of the blood brain barrier, and neuroinflammation. They may cause AD's neurodegeneration, cognitive and functional impairments, and neuropsychiatric symptoms.

Bilkei-Gorzo et al. (Nature Med, 2017 June, 23: 782-787) reported that a low dose of THC restores cognitive function in old mice. Three cohorts (“young” mice aged 2 month, “mature” mice aged 12 months, “old” mice aged 18 months) received a continuous release of either THC (3 mg/kg bodyweight per day) for the treatment group or vehicle only for the control group four weeks. Testing the three groups who were not treated with THC, mice in the mature and old groups performed worse in the different tasks than mice in the young group. In other words, the cohorts appeared to model an age-related decline in cognitive performance. Mice who were treated with THC demonstrated that young mice performed worse than the control group of young mice. This also reflects the observation of THC's detrimental effects on cognition in young animals and humans. For the mature and old groups of mice treated with THC for four weeks, their cognitive deficits in learning and memory were erased and they performed as well as the young group of mice. This reversal in the age-related decline in cognitive performance lasted long after treatment had terminated.

Cao (U.S. Ser. No. 11/065,225) provides a method for treating Alzheimer's disease by administering THC in an “ultra-low” amount. In one embodiment, THC is administered in an amount of at least 0.2 μg/kg of a patient's bodyweight. THC is administered in an amount from about 0.2 μg/kg to about 0.16 mg/kg of the patient's bodyweight, and preferably in an amount from about 0.2 μg/kg to about 0.02 mg/kg of the patient's bodyweight. In another embodiment, THC in an amount of at least 0.2 μg/kg of a patient's bodyweight is administered with melatonin from about 0.11 mg/kg to about 1.1 mg/kg of a patient's bodyweight. A ratio of THC to melatonin from about 1:400 to about 1:4000 is also taught.

Mukunda et al. (WO 2019/190608) provides methods and compositions for treating central nervous system disorders, such as Alzheimer's disease and Parkinson's disease, with a composition having THC from about 14 μg to about 10.0 mg (preferably from about 14 μg to about 2.0 mg) per 70-kg bodyweight, melatonin from about 1.4 mg to about 20.0 mg per 70-kg bodyweight, and curcumin from about 0.35 mg to about 500 mg per 70-kg bodyweight. In a preferred embodiment, CBD from about 14 μg to about 200 mg per 70-kg bodyweight is administered along with the specified amounts of THC and melatonin, with or without curcumin. In a further embodiment, a composition has THC from about 14 μg to about 2.0 mg per 70-kg bodyweight and CBD from about 14 μg to about 200 mg per 70-kg bodyweight. Another composition has CBD from about 14 μg to about 200 mg per 70-kg bodyweight and melatonin from about 1.4 mg to about 20.0 mg per 70-kg bodyweight. Additional compositions comprise THC or CBD, each with curcumin in the disclosed amounts without melatonin. Example 1 provides 45 mg THC as shatter, 45 mg melatonin, and 30 mg curcumin for making 30 ml of the formulation. Example 9 provides 45 mg THC, 45 mg melatonin, 30 mg turmeric, and 33 mg rutin in 30 ml. Example 10 provides 0.25% THC, 0.25% to 5% CBD, 0.15% melatonin, 0.05% curcumin, and 0.05% rutin in 30 ml. The effects of THC, melatonin, and curcumin on AD biomarkers and symptoms were discussed.

The public's interest in cannabis' beneficial effects, the availability of hemp-derived products with low amounts of THC, and caution about subjecting already-frail AD patients to the known risks of harm and addiction from THC has focused attention on research into new treatments on cannabis-derived drug products. Of all the plant's cannabinoids, it might seem counterintuitive to use THC to treat neuropsychiatric disorders because “[i]t is responsible for the characteristic effects of cannabis, such as euphoria, relaxation, and changes in perceptions. THC can also produce dysphoria, anxiety, and psychotic symptoms.” See Dos Santos et al. (Adv Exp Med Biol, 2020 December, 1264: 19-45). In controlled clinical trials, the efficacy of treatments using cannabis-derived products has been supported, at best, by weak evidence. Rigorous review of such treatments by the U.S. Food and Drug Administration (FDA) would be expected. See 85 FR 44305 (2020). “[T]he FDA has not approved a marketing application for cannabis for the treatment of any disease or condition. The agency has, however, approved one cannabis-derived drug product: Epidiolex (cannabidiol), and three synthetic cannabis-related drug products: Marinol (dronabinol), Syndros (dronabinol), and Cesamet (nabilone)” according to www.fda.gov/news-events/public-health-focus/fda-regulation-cannabis-and-cannabis-derived-products-including-cannabidiol-cbd. CBD was approved for the treatment of seizures associated with Lennox-Gastaut syndrome or Dravet syndrome. Dronabinol was approved for use in nausea associated with cancer chemotherapy and for the treatment of anorexia associated with weight loss in AIDS patients.

Pauli et al. (Front Pharmacol, 2020 February, 11: 63) reviewed 16 completed clinical trials involving CBD-based drugs and concluded, “CBD formulations have been tested in preclinical studies to have diverse medicinal properties, such as anti-nausea, anti-emetic, anti-tumor, anti-inflammatory, anti-depressant, anti-psychotic, and anti-anxiolytic; however, the variance in drug formulations used and limited sample sizes reduce the applicability of these studies in clinical applications.” Paunescu et al. (Am J Ther, 2020 May, 27: e249-e269) stated, “No clear conclusion can be drawn on the effectiveness of psychoactive cannabinoids in the treatment of psychiatric manifestations, in particular agitation and aggression, in AD” after meta-analysis of six clinical trials. Forty-six clinical trials were reviewed by Velayudhan et al. (PLoS Med, 2021 March, 18: e1003524) for safety and tolerability of cannabinoids in older adults.

van den Elsen et al. (Neurology, 2015 June, 84: 2338-2346) compared 4.5 mg/day THC to placebo. Neuropsychiatric symptoms improved in both treatment and placebo groups. No differences were shown for agitation or reduction from baseline of Neuropsychiatric Inventory (NPI) total score. Forester et al. (Alzheimers Dement, 2017 July, 13 Abstract Suppl: P3-026) and Rosenberg et al. (Am J Geriatric Psychiatry, 2020 April, 28: S121-5122) described recruit-ment for a pilot clinical trial of dronabinol in AD patients having severe agitation. See also NCT02792257. Outen et al. (Am J Geriatric Psychiatry, 2021 December, 29: 1253-1263) discussed the possible contributory mechanisms for agitation in AD and the possible therapeutic rele-vance of cannabinoids.

In a case report, Defrancesco & Hofer (Front Psychiatry, 2020 May, 11: 413) treated a patient having severe Alzheimer's disease with 4.9-6.7 mg dronabinol, 10 mg donepezil, and 50 mg sertraline daily because of “the known positive anxiolytic, pain-relieving and calming effect of cannabinoids.” There were improvements in her emotional state and behavioral symptoms (i.e., disruptive behavior, aggression, and sedation). The patient's NPI total score decreased from 20 to 15.

In a meta-analysis, Ruthirakuhan et al. (J Clin Psychiatry, 2019 January, 80: 18r12617) concluded, “The efficacy of cannabinoids on agitation and aggression in patients with AD remains inconclusive, though there may be a signal for a potential benefit of synthetic cannabinoids.” Cf. Liu et al. (CNS Drugs, 2015 August, 29: 615-623). Herrmann et al. (Am J Geriatric Psychiatry, 2019 November, 27: 1161-1173) compared nabilone to placebo in patients having moderate-to-severe Alzheimer's disease (NCT02351882). Daily dosage for nabilone was a maximum of 2 mg. It was suggested that nabilone may be an effective treatment for agitation, with potential benefits on neuropsychiatric symptoms and caregiver distress (NPI-NH total score and NPI-NH caregiver distress, respectively). In a systematic review, available online on the same day our priority application was being filed, Bosnjak Kuharic et al. (Cochrane Database Syst Rev, 2021 September, 9: CD012820) conclude from their meta-analysis of randomized controlled trials of cannabinoids for the treatment of dementia (126 participants) that “we cannot be certain whether cannabinoids [THC] have any beneficial or harmful effects on dementia.”

The ability to discriminate small but clinically significant changes in patients with mild cognitive deficits (e.g., MCI, amnestic MCI) or at an early stage of Alzheimer's disease (e.g., preclinical AD, prodromal AD, Stage 1 or 2 AD, early-stage AD, mild AD) is critical. Popular tests and rating scales used to screen for cognitive decline and dementia include the Alzheimer's Disease Assessment Scale-Cognitive Subscale (ADAS-Cog), Clinical Dementia Rating Scale Sum of Boxes (CDR-SOB), General Practitioner Assessment of Cognition (GPCOG), Memory Impairment Screen (MIS), Mini-Cog, Mini-Mental State Examination (MMSE), Montreal Cognitive Assessment (MoCA), and Six Item Cognitive Impairment Test. The high failure rate of new Alzheimer's disease treatments during phase III clinical trials has led regulatory authorities to reconsider their historic drug approval processes. See Riordan & Drosopoulou (J Clin Stud, 2018 April, 10: 20-22) and Sabbagh et al. (Alzheimers Dement, 2019 January, 5: 13-19).

In the literature cited above, the art showed its skepticism about the efficacy of cannabinoids for treating dementia, including mild cognitive impairment and early-stage AD, and caution about the possibility of THC toxicity because of its psychoactive activity, especially for elderly patients who are frail. Such toxicity has been addressed by reducing the dosage of THC to an ultra-low amount and/or specifying a generous ratio of CBD:THC (i.e., one or more). Here, a minor amount of CBD may be present in the inventors' formulation because the THC used is derived from cannabis plant material, CBD may not contribute substantially to treatment efficacy because its concentration is extremely low (i.e., less than 1% wt/vol).

Surprisingly, the inventors discovered intermediate dosages of THC and melatonin were efficacious in treating multiple neuropsychiatric symptoms of AD without requiring an entourage effect, CBD, and/or other non-THC cannabinoid compounds. This success may reflect a synergistic interaction between THC compounds and melatonin compounds for treating these clinical indications. Here, neurodegenerative diseases and neurologic disorders are treated, neuropsychiatric symptoms are alleviated or ameliorated, and the efficacy of intermediate dosages of THC and melatonin was shown in a clinical trial of AD patients.

### SUMMARY

An objective is to treat at least dementia, Alzheimer's disease (AD), familial AD, late-onset AD, preclinical AD, prodromal AD, Stage 1 AD, Stage 2 AD, early-stage AD, mild AD, mild cognitive impairment (MCI), amnestic MCI, major neurocognitive disorder, mild neurocognitive disorder, HIV associated dementia (HAD), amyloidosis, dementia-related psychosis, depression, major depressive disorder, an anxiety disorder, panic disorder, a sleep disorder, a neurodegenerative disease, or a neurologic disorder.

Another objective is to improve at least one symptom selected from delusions, hallucinations, agitation, depression, anxiety, irritability, aberrant motor behavior, sleep disorders, or eating disorders.

In an aspect, a product is provided (e.g., a daily dosage, a medicament, a medical device, or a kit of parts) for treating a subject in need of such treatment, comprising any effective amount of a tetrahydrocannabinol (THC) compound ranging from about 2.0 mg to about 7.0 mg (or in the range from about 0.0286 mg/kg to about 0.1000 mg/kg of the subject's bodyweight) and any effective amount of a melatonin compound ranging from about 1.0 mg to about 4.4 mg (or in the range from about 0.0143 mg/kg to about 0.0629 mg/kg of the subject's bodyweight). Cannabidiol (CBD) or another non-THC cannabinoid compound, such as cannabichromene (CBC), cannabigerol (CBG), or cannabinol (CBN), may or may not be present in the product. Thus, the product may not include a mixture of cannabinoid compounds (e.g., nabixomols). Alternatively, the product may not include more than about 2% (wt/vol) of CBD and/or other non-THC cannabinoid compounds.

In another aspect, a method is provided for treating a subject in need of treatment, comprising administering daily to a subject in need of treatment any effective amount of a tetrahydrocannabinol (THC) compound ranging from about 2.0 mg to about 7.0 mg (or in the range from about 0.0286 mg/kg to about 0.1000 mg/kg of the subject's bodyweight) and any effective amount of a melatonin compound ranging from about 1.0 mg to about 4.4 mg (or in the range from about 0.0143 mg/kg to about 0.0629 mg/kg of the subject's bodyweight). Daily administration of the THC compound and the melatonin compound to the subject may be continued for at least one week, at least two weeks, at least three weeks, at least one month, at least two months, at least three months, or at least six months, at least nine months, or at least one year. Cannabidiol (CBD) or another non-THC cannabinoid compound (CBC, CBG, CBN) may or may not be administered. A cannabis extract containing a mixture of cannabinoid compounds, such as nabixomols, may or may not be administered. For example, less than about 2% (wt/vol) of CBD and/or other non-THC cannabinoid compounds may be used for treatment.

The THC compound may be in any amount ranging from about 2.25 mg to about 6.75 mg (or in the range from about 0.0321 mg/kg to about 0.0964 mg/kg of a subject's bodyweight). The THC compound may be in any amount ranging from about 2.5 mg to about 6.5 mg (or in the range from about 0.0357 mg/kg to about 0.0929 mg/kg of a subject's bodyweight). The THC compound may be in any amount ranging from about 2.75 mg to about 6.25 mg (or in the range from about 0.0393 mg/kg to about 0.0893 mg/kg of a subject's bodyweight). The THC compound may be in any amount ranging from about 3.0 mg to about 6.0 mg (or in the range from about 0.0429 mg/kg to about 0.0857 mg/kg of a subject's bodyweight).

A THC compound may be dronabinol, nabilone, trans-delta-8-THC (Δ8-THC), cis-Δ8-THC, trans-delta-9-THC (Δ9-THC), cis-Δ9-THC, one or more Δ9-THC stereoisomers thereof (e.g., either or both of each enantiomeric pair), Δ9-THC carboxylic acid (THCA), Δ9-THC-4-oic acid (THCA-A), Δ9-THC-2-oic acid (THCA-B), 11-hydroxy-Δ9-THC (11-OH-THC), 11-nor-9-carboxy-Δ9-THC (11-COOH-THC), anandamide, 2-arachidonoyl-glycerol, 2-arachido-noyl-glyceryl ether, or any combination thereof. A THC compound may be selected from any one, two, four or all of the following: 7,8,9,10-tetrahydro-6,6,9-trimethyl-3-pentyl-6H-dibenzo [b,d]pyran-1-ol (Δ6a, 10a-THC); (9R,10aR)-8,9,10,10a-tetrahydro-6,6,9-trimethyl-3-pentyl-6H-dibenzo[b,d]pyran-1-ol (Δ6a, 7-THC); (6aR,9R,10aR)-6a,9,10,10a-tetrahydro-6,6,9-tri methyl-3-pentyl-6H-dibenzo[b,d]pyran-1-ol (Δ7-THC); 6a,7,8,9-tetrahydro-6,6,9-trimethyl-3-pentyl-6H-dibenzo[b,d]pyran-1-ol (Δ10-THC); (6aR,10aR)-6a,7,8,9,10,10a-hexahydro-6,6-di methyl-9-methylene-3-pentyl-6H-dibenzo[b,d]pyran-1-ol (Δ9, 11-THC). More generally, a THC compound may or may not be an endocannabinoid, a precursor in THC biosynthesis, a THC metabolite, or a synthetic agonist of a cannabinoid receptor (CB₁ or CB₂ receptor). If the THC compound's molecular weight is significantly different from Δ⁹-THC, the THC compound's amount may be adjusted for its bioavailability and equivalent cannabinoid receptor signaling activity as compared to Δ⁹-THC.

The melatonin compound may be in any amount ranging from about 1.25 mg to about 4.20 mg (or in the range from about 0.0179 mg/kg to about 0.0600 mg/kg of a subject's bodyweight). The melatonin compound may be in any amount ranging from about 1.5 mg to about 4.0 mg (or in the range from about 0.0215 mg/kg to about 0.0571 mg/kg of a subject's bodyweight). The melatonin compound may be in any amount ranging from about 1.75 mg to about 3.75 mg (or in the range from about 0.0250 mg/kg to about 0.0536 mg/kg of a subject's bodyweight). The melatonin compound may be in any amount ranging from about 2.0 mg to about 3.5 mg (or in the range from about 0.0286 mg/kg to about 0.0500 mg/kg of a subject's bodyweight).

A melatonin compound may be circadin, N-acetyl-5-methoxytryptamine, agomelatine, ramelteon, tasimelteon, 5-methoxytryptamine, N-acetyl-serotonin, or any combination thereof. More generally, a melatonin compound may or may not be a precursor in melatonin biosynthesis, a synthetic agonist of a melatonin receptor (MT₁ or MT₂ receptor), or a neurotransmitter. If the melatonin compound's molecular weight is significantly different from N-acetyl-5-methoxytryptamine, the melatonin compound's amount may be adjusted for its bioavailability and equivalent melatonin receptor signaling activity as compared to N-acetyl-5-methoxytryptamine.

Compositions and/or products may further comprise a curcumin-related compound. Likewise, methods may further comprise administering a curcumin-related compound in any amount ranging from about 0.3 mg to about 1.5 mg (or in the range from about 0.0043 mg/kg to about 0.0215 mg/kg of a subject's bodyweight), or ranging from about 0.6 mg to about 1.2 mg (or in the range from about 0.0086 mg/kg to about 0.0171 mg/kg of a subject's bodyweight), or ranging from about 0.8 mg to about 1.0 mg (or in the range from about 0.0114 mg/kg to about 0.0143 mg/kg of a subject's bodyweight). The curcumin-related compound may be curcumin, moreover it may or may not be turmeric, curcuminoids other than curcumin, structural derivatives of curcumin, analogs of curcumin that are enhanced for at least better absorption, bioavailability, stability, or taste, or any combination thereof.

Compositions and/or products may further comprise a rutin-related compound. Likewise, methods may further comprise administering a rutin-related compound in any amount ranging from about 0.3 mg to about 1.5 mg (or in the range from about 0.0043 mg/kg to about 0.0215 mg/kg of a subject's bodyweight), or ranging from about 0.6 mg to about 1.2 mg (or in the range from about 0.0086 mg/kg to about 0.0171 mg/kg of a subject's bodyweight), or ranging from about 0.8 mg to about 1.0 mg (or in the range from about 0.0114 mg/kg to about 0.0143 mg/kg of a subject's bodyweight). The rutin-related compound may be rutin, moreover it may or may not be quercetin glycosides other than rutin (e.g., quercetin), rutinosides other than rutin (e.g., hesperidin), structural derivatives of rutin, analogs of rutin that enhanced for at least better absorption, bioavailability, stability, or taste, or any combination thereof.

Compositions and/or products may further comprise one, two, or more polyphenolic compounds. Alternatively, one, two, or more polyphenolic compounds may be provided separately from THC and melatonin compounds in compositions (e.g., two, three, or four distinct compositions) or products (e.g., positioned at distinct locations within a device or a kit of parts). Likewise, methods may further comprise administering one, two, or more polyphenolic compounds. They may be synchronously administered with at least a THC compound and/or a melatonin compound, or separately administered. The polyphenolic compounds may be selected from turmeric, curcuminoids, curcumin, curcumin-related compounds, quercetin glycosides, rutin, rutinosides, rutin-related compounds, or any combination thereof. A Vitamin E-related compound may be included to enhance effectiveness or stability.

Compositions and/or products may further comprise a Vitamin E-related compound. Likewise, methods may further comprise administering a Vitamin E-related compound in any amount equivalent to an amount of alpha-tocopherol ranging from about 20 mg to about 55 mg (or in the range from about 0.286 mg/kg to about 0.786 mg/kg of a subject's bodyweight), ranging from about 30 mg to about 45 mg (or in the range from about 0.429 mg/kg to about 0.643 mg/kg of a subject's bodyweight), or ranging from about 35 mg to about 40 mg (or in the range from about 0.500 mg/kg to about 0.571 mg/kg of a subject's bodyweight). The Vitamin E-related compound may be synthetic Vitamin E (e.g., dl-alpha-tocopherol), natural Vitamin E derived from a plant oil (e.g., olive, sunflower, corn, soybean), tocopherols (E306), alpha-tocopherol (e.g., d-alpha-tocopherol), beta-tocopherol, gamma-tocopherol (e.g., d-gamma-tocopherol), delta-tocopherol, alpha-tocopherol and beta-tocopherol, alpha-tocopherol and gamma-tocopherol, structural derivatives of tocopherol (e.g., salts, esters), analogs of tocopherol that enhanced for at least better absorption, bioavailability, stability, or taste, or any combination thereof.

A vehicle and/or a carrier may be present in at least the compositions or the products, or at least administered in the methods. At least some of the THC compound, at least some of the melatonin compound, or at least some of both may be solubilized in an alcohol (e.g., ethanol), an alcoholic water (e.g., about 5%-20% ethanol in water), an oil (e.g., about 70% or greater fatty acid), an emulsion (e.g., a nanoemulsion), a lipid bilayer (e.g., liposomes, nanoparticles), or any combination thereof to enhance the compound's stability and solubility.

An excipient may also be present in at least the compositions or the products, or at least administered in the methods. The excipient may be one or more selected from preservatives (e.g., ascorbic acid, ethanol, honey), emulsifiers (e.g., polysorbate 80, alpha-tocopheryl PEG 1000 succinate or TPGS 1000), or any combination thereof. Other examples of excipients are listed elsewhere in this specification or are known in the art.

In any of the foregoing methods, the subject may have been diagnosed prior to treatment with at least dementia, Alzheimer's disease, major neurocognitive disorder, mild cognitive impairment, mild neurocognitive disorder, dementia-related psychosis, depression, major depressive disorder, minor depressive disorder, an anxiety disorder, panic disorder, a sleep disorder, or an eating disorder. Diagnostic criteria are described in the Diagnostic and Statistical Manual of Mental Disorders (DSM) and the International Statistical Classification of Diseases and Related Health Problems (ICD).

In any of the foregoing methods, treatment may improve at least a cognitive skill including, but not limited to, memory, learning, attention, information processing speed, executive function, or any combination thereof. Cognitive deficits may be assessed by art-recognized tests and rating scales: Mini-Mental State Exam (MMSE), Mini-Cog test, Alzheimer's Disease Assessment Scale (ADAS-cog14), other tests that measure immediate logical memory, delayed logical memory, digit span, visual memory span tests including Wechsler Memory Scale Revised (WMS-R), block-design, and digit-symbol coding tests such as Wechsler Adult Intelligence Scale-3rd (WAIS-III), Stroop test, Trail Making Test (TMT), Montreal Cognitive Assessment (MoCA), and Neuropsychological Test Battery (NTB) for cognitive function, among others. See also those listed in Baldwin & Farias (Curr Protoc Neurosci, 2009 October, 49: 20.3.1-20.3.8) and Robert et al. (Alzheimer Res Ther, 2010 August, 2: 24).

In any of the foregoing methods, the subject may experience one or more symptoms that are behavioral changes of Alzheimer's disease including, but not limited to, delusions, hallucinations, agitation/aggression, depression, anxiety, elation/euphoria, apathy, disinhibition, irritability, aberrant motor behavior, sleep disorders, appetite/eating disorders, or any combination thereof. Behavioral changes may be assessed by art-recognized tests and rating scales such as, for example, the Behavior Rating Scale for Dementia, Behavioral Pathology in Alzheimer's Disease Rating Scale, Neurobehavioral Rating Scale, and Neuropsychiatric Inventory. Treatment may or may not decrease severity of at least one, at least two, at least three, or at least four symptoms. At least one symptom may or may not be decreased in severity within one week, within two weeks, within three weeks, or within one month of starting treatment.

In any of the foregoing methods, a caregiver of the subject may be distressed prior to treatment by at least one, at least two, at least three, or at least four of the subject's symptoms. The caregiver's distress may or may not be decreased within one week, within two weeks, within three weeks, or within one month of starting treatment.

In any of the foregoing methods, the subject's ability to metabolize THC may be assayed prior to treatment by identifying genetic polymorphisms in at least CYP2C9, CYP3A4, CYP2C19, CYP1A1, CYP1A2, or any combination thereof. The effective amount of THC may be increased or decreased based on the subject's metabolism as predicted from expression of one or more of the aforementioned cytochrome P450 isoenzymes.

In any of the foregoing methods, the subject may have been medicated prior to treatment with one or more drugs from the following: antidepressants, antipsychotics, mood stabilizers, selective serotonin reuptake inhibitors, serotonin/norepinephrine reuptake inhibitors, or anxiolytics. The drug's daily dosage may or may not be decreased within one week, within two weeks, within three weeks, or within one month of starting treatment.

In any of the foregoing methods, the subject may or may not have been medicated prior to treatment with at least aducanumab, donepezil, galantamine, memantine, rivastigmine, solanezumab, or any combination thereof. If so medicated, at least aducanumab, donepezil, galantamine, memantine, rivastigmine, or solanezumab may or may not be administered to the subject after one week, after two weeks, after three weeks, after one month, after two months, after three months, after six months, after nine months, or after one year of treatment. Alternatively, an effective amount of at least aducanumab, donepezil, galantamine, memantine, rivastigmine, or solanezumab may or may not be reduced from the amount taken by the subject before treatment after one week, after two weeks, after three weeks, after one month, after two months, after three months, after six months, after nine months, or after one year of treatment.

In any of the foregoing methods, the effective amounts of at least the THC and melatonin compounds may be administered once and only once per day. Alternatively, about 40%-60% (e.g., about half) of the effective amount of at least the THC compound, at least the melatonin compound, or both is administered twice per day (e.g., morning and evening).

In any of the foregoing methods, the subject may be treated every day for at least one week, at least two weeks, at least three weeks, at least one month, at least two months, at least three months, at least six months, at least nine months, or at least one year.

Exemplary ranges for amounts or concentrations that may be administered in a daily dosage are described herein. For convenience, while the daily dosage of THC and melatonin compounds may be administered in a single dose once per day, the daily dosage may be divided into a plurality of smaller doses that are all administered within about 24 hours (e.g., 22-26 hours). If there are two doses per day, complementary doses may contain about 40%-60% (e.g., about half) of the daily dosage for each compound totaling 100% for both doses; alternatively, the entire daily dosage of THC compound may be administered once at a convenient time (e.g., morning) and the entire daily dosage of the melatonin compound may be administered once at a distinct time (e.g., evening). Two doses may be separately administered twice per day, possibly at times separated by about 12 hours (e.g., 10-14 hours) or at least about 8 hours. Three doses may be separately administered thrice per day, possibly at times separated by about 8 hours (e.g., 7-9 hours) or at least about 6 hours.

Modifications and variations of the invention described above are within the skill of a person skilled in the relevant arts. Therefore, obvious alternatives to the claimed invention are protected under the doctrine of equivalents. The following detailed description and examples are provided to aid in the public's understanding of the invention. Nor do they do not limit the scope of the appended claims unless a limitation is imported from the specification and explicitly recited in the claims.

Specific embodiments, which are examples contemplated to apply one or more principles of the inventors' discovery and invention, are described below. Such examples are not necessarily required to practice the improvement discovered by the inventors.

### DETAILED DESCRIPTION OF SPECIFIC EMBODIMENTS

Certain embodiments and examples that were accomplished by the inventors are described below. Other embodiments and examples applying aspects of their invention are contemplated, but it is not believed that operability of every contemplated embodiment or example is necessarily required to benefit from any of the improvements discovered by the inventors.

Unless defined otherwise herein, technical terms have the same meaning as would be understood by a person skilled in the medical arts related to the invention (i.e., geriatrician, neurologist, or psychiatrist). Depending on the technology, the person may also have skill (i.e., knowledge, training, and experience) in chemistry, pharmacology, physiology, and biostatistics.

The terms “a” and “an” are defined as “at least one” and “one or more” with them being recited interchangeably herein. They include both singular and plural forms of the article's noun unless either form contradicts the context in which the noun is used. As used herein, they are equivalent to “one, more than one, either, or both” or “once, more than once, either, or both” and they may be limited in an amendment by substituting “only one” or “only once” to exclude a plurality (e.g., two or twice, three or thrice, etc.) and “more than one” or “more than once” to exclude the singularity. Similar appearing terms like single, unique, several, multiple, plurality, etc. have their ordinary meanings. Furthermore, recitation of a pronoun herein includes both singular and plural forms of the pronoun (one person and a plurality of people), without necessarily implying limitation to “only one” or “more than one” unless contradicted by the context in which the pronoun is used.

When a range is recited herein for a function or a property (e.g., age, amount, chemical formula, concentration, density, molecular weight, temperature), all combinations of ranges (e.g., A to D and C to G imply A to G), subcombinations of subranges (e.g., A to C, C to D, D to G), and specific embodiments therein (e.g., A, B, C, D, E, F, G) are also part of the following description of the invention. As used herein, the term “about” when used in the context of a numeric value or a numeric range means that the value or range may deviate by an amount that is understood by a person skilled in the art to be reasonable under the circumstances, e.g., within the variability of measurement and observation (random and systematic experimental errors, respectively). Alternatively, a numeric value or a numeric range can vary by 10% of the value or the range's limits. For example, “about 23° C.” can mean a temperature in the range 30 from 20.7° C. to 25.3° C. (or 23° C.±2.3° C.) for the thermometer's experimental error while “about 21-25° C.” can mean a temperature in the range from 18.9° C. to 27.5° C.

The terms “comprising” and “comprised of” are defined as requiring at least the acts or elements named thereafter, but other acts/elements that were not mentioned may or may not also occur/be present. In other words, a process or product comprising A requires at least A, but does not imply exclusion of B merely because B is not mentioned. Wherever “comprising” or “comprised of” is recited in this specification, “containing” or “including” or “contained” or “included in” may be substituted to support amendment of a claim. Wherever “containing” or “including” or “contained in” or “included in” is recited in this specification, “comprising” or “comprised of” may be substituted to support amendment of an appended claim. Listing an act/element as “optionally” or “optional” means it may or may not occur/be present.

Wherever “comprising,” “comprised of,” “containing,” or “including” is recited in this specification, the term “consisting of” or “consists of” may be substituted to support amendment of an appended claim. The terms “consisting of” and “consists of” are defined as requiring only the acts or objects named thereafter, excluding the occurrence/presence of other acts or objects that were not mentioned.

Wherever “comprising,” “comprised of,” “containing,” or “including” is recited in this specification, the term “consisting essentially of” or “consists essentially of” may be substituted to support amendment of an appended claim. The terms “consisting essentially of” and “consists essentially of” for a process or product are defined as requiring at least the named acts or objects, as well as other acts or objects that were not mentioned may or may not also occur/be present if their occurrence/presence does not materially affect the basic and novel characteristics of the process or product.

A “device” is comprised of a physical structure (e.g., a dispenser) and a composition (e.g., medicament), which contains at least two, three, four, five, or more compounds as described herein (e.g., a THC compound and a melatonin compound, optional polyphenolic compounds such as a curcumin-related compound and a rutin-related compound, optional Vitamin E-related compound, optional at least one chemotherapeutic agent), such that effective amounts of the at least two, three, four, five, or more compounds for treating a subject are dispensed. A process for manufacturing “a device” involves at least associating the composition with the physical structure: e.g., loading the composition into the reservoir of a dispenser. Examples of a dispenser include, but are not limited to, dressings and patches, droppers and pipets, enema bottles, hypodermic syringes, implanted depots and pumps, infusion pumps, inhalers, injection ampules and vials, insufflators, intravenous (IV) drips with cannula/catheter, misters and sprayers, nebulizers, pill bottles, pillboxes, suppositories, and vaporizers.

A “dose” is a quantity (e.g., an amount by mass or within a range of masses) of a medicament, a therapeutically active compound, or a food ingredient that is at least given to or taken by a subject at each administering step. A “unit dose” is a dose in its physical form (i.e., solid, semisolid, or liquid) as described herein. A “dosage” is a dose (e.g., an amount by mass or within a range of masses) of a medicament, a therapeutically active compound, or a food ingredient or the number of doses administered over a time period (e.g., once per day or daily, twice per day such as in the morning and evening, once per eight hours or every eight hours, once per 12 hours or every 12 hours). Alternatively, the frequency during a single day may be once or twice daily (e.g., morning and evening, with or without a meal). It would be equivalent to describe the quantity of a dose or a dosage as a concentration (e.g., a mass percentage or a mass per volume) and a volume, whose product is equal to the quantity. A “single unit dose” is a dosage in its physical form (i.e., solid, semisolid, or liquid), such as, for example, ampule, capsule, chartula or sachet, chewable candy or gum, disintegrating strip or cap, lozenge or pastille, tablet, or vias. The quantity is for an adult human (male or female) adjusted for weight (e.g., adult human of 70-kg bodyweight) or, in some embodiments, irrespective of weight (e.g., any adult human).

A “medicament” is a pharmaceutical composition (more particularly, a unit dose, especially a daily dosage) comprised of at least two, three, four, five, or more compounds as described herein and pharmaceutically-acceptable excipients. The “medicament” comprises the THC compound and the melatonin compound, optional polyphenolic compounds, optional Vitamin E-related compound, and optional at least one chemotherapeutic agent. The “medicament” may be manufactured by formulating at least THC and melatonin compounds as described herein, at least optional polyphenolic compounds, at least optional Vitamin E-related compound, at least pharmaceutically-acceptable excipients, at least optional vehicle, and at least optional carrier in the same formulation. Examples of suitable excipients include, but are not limited to, preservatives, antimicrobials, antioxidants, stabilizers, emulsifiers, surfactants, solubilizers, sweeteners and other flavoring agents, food dyes and other coloring agents, essential oils and other odor masking agents.

The terms “effective amount” and “therapeutically effective amount” refer to an amount of at least two, three, four, five, or more compounds (individually or as the sum) that is sufficient to treating a subject, who receives a beneficial or therapeutic effect. The terms may refer to one or more doses administered to a subject within a time period (i.e., total dose), especially a dose administered once and only once per day (i.e., daily dosage). The amount may be adjusted based on professional judgment, the disease's current symptoms and severity, the route and type of administration, any other compounds that are co-administered, and the subject's characteristics including, but not limited to, age, comorbidities and their current symptoms, family history, fitness, gender at birth, genetic profile, racial background, and weight. The total dose or daily dosage is easily recalculated after adjustment.

The term “treatment” refers to treating a subject having a cognitive or perceptual deficit, a neuropsychiatric symptom, a neurodegenerative disease, a neurologic disorder, or any combination thereof for the benefit of the treated subject. Generally, a step of administering one or a mixture of chemical or biological compounds to a subject is a hallmark of methods for treatment. Treatment encompasses “therapeutic” treatments, “prophylactic” treatments, “preventive” treatments, “palliative” treatments, or any combination thereof. The subject may or may not be diagnosed with at least dementia, mild cognitive impairment (MCI), amnestic MCI, Alzheimer's disease (AD), early-onset AD, late-onset AD, sporadic AD, familial AD, preclinical AD, prodromal AD, early-stage AD, mild AD, Stage 1 AD, Stage 2 AD, or any combination thereof. An objective of “therapeutic treatment” may be to enhance health or wellness, promote at least a cognitive or a functional improvement, reduce at least the frequency or the severity of a symptom caused by disease, or any combination thereof for the treated subject. Treatment might continue until complete and full recovery, although this might never be achieved for a chronic or fatal condition. An objective of “prophylactic treatment” may be to decrease the risk of at least occurrence of disease, at least relapse of disease in the future, at least recurrence of a symptom caused by disease, or any combination thereof for the treated subject. An objective of “preventive treatment” may be to avoid at least deterioration of health or wellness caused by disease, slow at least development of disease, halt at least the worsening of frequency or severity of a symptom caused by disease, or any combination thereof for the treated subject. An objective of “palliative treatment,” which may be without curative intent, is to ameliorate a symptom caused by disease, distract from at least stress or pain caused by disease, relieve at least discomfort or pain caused by disease, encourage happiness, extend life expectancy, or any combination thereof for the treated subject. Achieving any one or more of these objectives may be considered a “therapeutic effect” of treatment. It would be understood by a person skilled in the art that achieving a beneficial or therapeutic effect may be difficult to demonstrate for any particular subject but might be demonstrable statistically in a cohort of similar subjects. As is known in the art, a beneficial or therapeutic effect of treatment may be achieved without necessarily curing disease or completely preventing disease.

Similarly, the term “therapy” refers to therapeutic treatment, prophylactic treatment, preventive treatment, palliative treatment, or any combination thereof as described in the last paragraph, which is incorporated by reference herein, for treating a subject in need thereof and who may or may not have been diagnosed prior to treatment with at least dementia, major neurocognitive disorder, mild cognitive impairment (MCI), amnestic MCI, mild neurocognitive disorder, Alzheimer's disease (AD), early-onset AD, late-onset AD, sporadic AD, familial AD, preclinical AD, prodromal AD, early-stage AD, middle-stage AD, late-stage AD, mild AD, moderate AD, severe AD, Stage 1 AD, Stage 2 AD, Stage 3 AD, Stage 4 AD, HIV associated dementia (HAD), amyloidosis, dementia-related psychosis, depression, major depressive disorder, dysthymia or persistent depressive disorder, minor depressive disorder, an anxiety disorder, panic disorder, a sleep disorder, an eating disorder, or any combination thereof. Generally, “therapy” is a method comprised of at least administering an effective amount of one or a mixture of chemical or biological compounds to a subject in need thereof. An objective of “therapy” is to obtain any one or more of the beneficial or therapeutic effects as described in the preceding paragraph, which is incorporated by reference herein. As is known in the art, a beneficial or therapeutic effect may be obtained without necessarily curing disease or completely preventing disease.

The term “diagnosis” refers to diagnosing a subject as having a cognitive or perceptual deficit, neuropsychiatric symptom, neurodegenerative disease, neurologic disorder, or any combination thereof. The diagnostician deciding (or forming a professional judgment) to diagnose the subject may be a physician, psychiatrist, or psychologist; possibly, board certified in neurology, geriatric psychiatry, or neuropsychology. A diagnosis of dementia requires a comprehensive evaluation: e.g., physical examination, psychiatric evaluation, psychological assessment, brain imaging, or any combination thereof. The diagnosis may also include a recommendation for additional test assaying, a proposed therapy, or options for treatment. A subject may consent to physical examination, psychiatric evaluation, psychological assessment, brain imaging, or any combination thereof, which are typically noninvasive and do not require submitting a sample for test assaying. Alternatively, a subject may consent to providing a sample, typically a body fluid or tissue, and the sample is collected and submitted for test assaying, e.g., genetic testing, neuropathological testing, genetic testing, neuropathological testing, pathogen culturing or detection, other laboratory test assays, or any combination thereof.

Generally, a test assaying step is a hallmark of methods for diagnosis (possibly along with an optional analyzing step, an optional comparing step, an optional determining step, or any combination thereof). An administering step of a method for treatment may come before or after test assaying (e.g., physical examination, psychiatric evaluation, psychological assessment, brain imaging, genetic testing, neuropathological testing, pathogen culturing or detection, other laboratory test assays such as blood counting and protein assays). While a sample may be collected from a subject during treatment, collecting the sample can be considered a step of a method for diagnosis, occurring before a test assaying step. Often, however, samples may be collected by subjects themselves or their caregivers outside of methods for treatment or diagnosis, the sample is then submitted for test assaying. But merely “acquiring” or “obtaining” a sample from a subject, “providing” or “sending” a sample to diagnose a subject, or “requesting” or “submitting” a sample for test assaying may or may not be considered a step of either methods for treatment or diagnosis in a hybrid method claim comprising, inter alia, both administration and test assaying steps. The test assaying of a sample from a subject will generate a result; generally, the result is information about a characteristic of the subject; the information may be analyzed to determine a diagnosis; and the diagnosis may be used, without limitation, to make decisions about treatment, to evaluate treatment, to confirm efficacy of treatment, to monitor changes in the disease (e.g., remission, relapse) due to treatment. A neurologist, psychiatrist, or psychologist may examine and analyze a subject to determine a diagnosis. But the test assaying step and the analyzing and/or the determining steps might be performed by a different person. A clinical testing laboratory or a hospital facility (e.g., radiology department) might perform the test assaying step and information resulting therefrom might be analyzed by another clinician (e.g., pathologist reading a stained slide, radiologist reading a PET-FDG image), who provides a report to and works with a diagnostician or a clinician treating a subject.

A “kit of parts” is comprised of a plurality of components, an optional dispenser of at least one or more components, a package, an optional seal that at least resists tampering with the components, an optional package insert, an optional expiration date, and an optional wrapper. One or more of the components may be used to perform at least one step of a method for treatment (i.e., “a medical aid kit”), at least one step of a method for diagnosis (i.e., “a test assay kit”), or at least one or two steps of a method for diagnosis and treatment (i.e., “a hybrid kit”). Generally, regarding the plurality of components, a medicament or pharmaceutical compounds are hallmarks of medical aid kits while a diagnostic reagent is a hallmark of test assay kits. For hybrid kits, a medicament and a diagnostic reagent would not be secured in the same compartment of a kit.

The terms “protocol” and “system” refer broadly to the invention as a whole (e.g., methods for treatment or diagnosis, products used in the methods, and processes for making those products; all sharing an inventive concept and having the objective of improving at least one behavioral or neuropsychiatric symptoms, at least one cognitive or functional impairment, any combination thereof, or otherwise benefiting a subject in need of treatment). They are distinguished by the protocol's inventive concept relating to a possible principle of operation (e.g., coordinating signaling by the cannabinoid receptors, the melatonin receptors, and other signaling pathways with which they interact to maintain or restore homeostasis; synergy of any two or more therapeutically active compounds), albeit treatment might not require binding to and signaling at cannabinoid and melatonin receptors, and/or the system's inventive concept relating to an element (e.g., combination of THC and melatonin compounds that are ligands for at least cannabinoid receptors and melatonin receptors; synergy between THC and melatonin compounds).

Here, the “subject” being treated is a human patient, male or female, because of the emphasis on functional and behavioral outcomes in a clinical trial. Relevant animal models for dementia are only capable of assessment for cognitive dysfunction or being assayed for biomarker changes. The subject may be an adult (i.e., 18 years or greater), a younger adult (i.e., from 18 years to 50 years), an older adult (i.e., greater than 50 years), or an elderly adult (i.e., 60 years or greater). A subject has early-onset Alzheimer's disease if diagnosed before the age of 65 years or late-onset Alzheimer's disease if diagnosed before at the age of 65 years or greater.

Furthermore, although the patient is the subject being treated, the patient's caregiver may be the recipient of a beneficial effect from treatment. A “caregiver” may be related to the patient as a family member or a friend prior to the disease's onset or the patient's treatment, especially for mild cognitive impairment or an early stage of disease when the patient is residing at home. During later stages, the caregiver may be a professional trained/experienced in (or even certified for) providing care for dementia or psychiatric patients.

Drugs approved for treating at least dementia, Alzheimer's disease, depression, anxiety disorders, panic disorder, or sleep disorders may or may not be used in combination with the protocols and systems described herein. Such drugs include, but are not limited to, selective serotonin reuptake inhibitors (e.g., citalopram, escitalopram, fluvoxamine, sertaline, vortioxetine); serotonin/norepinephrine reuptake inhibitors (e.g., desvenlafaxine, duloxetine, levomilnacipran, venlafaxine); antidepressants (e.g., mirtazapine, trazodone); mood stabilizers (e.g., carbamazepine, gabapentin, lamotrigine, oxcarbazepine topiramate, valproic acid); antipsychotics (e.g., aripiprazole, haloperidol, olanzapine, perphenazine, quetiapine, risperidone, ziprasidone); anxiolytics (e.g., buspirone, gepirone, ipsapirone, tandospirone); and orexin receptor antagonists (e.g., lemborexant, suvorexant). Some drugs treat multiple symptoms. Some drugs may or may not be avoided, including anticholinergics, benzodiazepines, and hypnotics (e.g., zolpidem, zopiclone, zaleplon), in compositions and methods for treating a subject.

Tetrahydrocannabinol (THC) can be chemically synthesized (i.e., artificial or synthetic THC) or purified from dried buds, flowers, seed, leaves, etc. of plants such as Cannabis sativa, Cannabis indica, and hybrid strains of Cannabis sativa and Cannabis indica. Cannabis plants and plant parts with high THC content are preferred. Separation by chromatography, dehydra-tion or distillation under vacuum, differential extraction, precipitation, recrystallization, solvent partitioning, or any combination thereof may be used to isolate THC from plant material as an alternative to chemical synthesis of pure THC. THC-containing, dry plant material from Cannabis may be extracted with solvent (e.g., carbon dioxide, N-butane, ethanol). Extract and solvent can be separated under vacuum using a rotary evaporator, falling film evaporator, or vacuum distillation (e.g., short-path distillation, fractional distillation, wiped film distillation). THC-containing extract is collected; optionally, the crude extract is defatted and dewaxed by winterization with cold ethanol to solidify fats and waxes, then filtered to remove solids from the extract before residual solvent is removed by distillation. Tetrahydrocannabinolic acid (THCA) can be converted to THC by decarboxylation (e.g., heating the dry plant material or the winterized extract) to increase the final yield of THC. THC distillate may be further refined, increasing THC purity and eliminating contaminants.

THC can be purified from cannabis plant material using commercially-available systems for extraction (e.g., closed loop extractors) and separation (e.g., vacuum distillation). The Transformer 1500-20L system made by Apeks Supercritical (Johnstown, Ohio) uses subcritical or supercritical carbon dioxide as the solvent to extract plant material. Extraction can cycle between supercritical (higher temperature and pressure) and subcritical (lower temperature and pressure) conditions, pooling the extracts because different compounds are extracted. Solvent is removed from the extract by releasing the pressure and having the gas boil off. Distillate is collected with cold separation. Delta Separations (Cotati, Calif.) supplies the CUP extraction unit to extract plant material with chilled ethanol using mechanical agitation and centrifugation; the FFE falling film evaporator unit for removing ethanol from the extract; and the RFD rolled film distillation unit for minimizing contact with the hot evaporative surface and collecting distillate on the condensing surface. IO Extractor 2.0 made by Luna Technologies (Portland, Oregon) uses hydrocarbons (i.e., butane and/or propane) as the solvent to extract plant material, resulting in a hash or honey oil that may be post-processed to purge residual solvent.

Artificial (pure) THC made by chemical synthesis and purified THC isolated from plants (i.e., THC isolate), which may be in solid form (i.e., amorphous or crystalline) and may be substantially free of cannabidiol (CBD), would be suitable for administering to a subject in need of treatment. Thus, THC can be provided as THC crystals. In a crude extract or a similar cannabis preparation having a mixture of cannabinoid compounds, THC may be purified away from other cannabinoid compounds (e.g., CBD) by separating and isolating them from each other using chromatography, fractional distillation, recrystallization, or any combination thereof to provide THC distillate that is greater than 80% pure, greater than 85% pure, greater than 90% pure, greater than 97% pure, greater than 98% pure, or greater than 99% pure. THC distillate as described in the last two paragraphs, and THC concentrates, such as, without limitation, amber glass or shatter (a solid form), budder or wax (a semisolid form), diamonds (i.e., crystalline), dust (i.e., powder), and hash or honey oil (a liquid form). A solid form of THC can be ground into a powder and used in an anhydrous composition.

A “THC compound” may be one or more of, without limitation, dronabinol, nabilone, trans-delta-9-THC (Δ⁹-THC), an isomer thereof (e.g., trans-delta-8-THC or Δ⁸-THC), Δ⁹-THC carboxylic acid (THCA), Δ⁸-THC carboxylic acid, Δ⁹-THC-4-oic acid (THCA-A), Δ⁸-THC-4-oic acid, Δ⁹-THC-2-oic acid (THCA-B), Δ⁸ THC-2-oic acid, 11-hydroxy-Δ⁹-THC (11-OH-THC), 11-hydroxy-Δ⁸-THC, 11-nor-9-carboxy-Δ⁹-THC (11-COOH-THC), 11-nor-9-carboxy-Δ⁸-THC, a cannabinoid receptor agonist (e.g., canbisol, levonantradol, arachidonyl-2′-chloro-ethylamide, AM-087, AM-411, AM-2389, CP 47,497, CP 55,940, HU-210, HU-308, JWH-018, JWH-048, JWH-133, O-1812, WIN 55,212-2), an endocannabinoid (e.g., N-arachidonoyl-ethanolamide, 2-arachidonoyl-glycerol, 2-arachidonoyl-glyceryl ether), or any combination thereof. A THC compound may be selected from any one, two, four or all of the following: 7,8,9,10-tetrahydro-6,6,9-trimethyl-3-pentyl-6H-dibenzo[b,d]pyran-1-ol (Δ6a, 10a-THC); (9R,10aR)-8,9,10,10a-tetrahydro-6,6,9-trimethyl-3-pentyl-6H-dibenzo[b,d]pyran-1-ol (Δ6a, 7-THC); (6aR,9R,10aR)-6a,9,10,10a-tetrahydro-6,6,9-trimethyl-3-pentyl-6H-dibenzo[b,d]-pyran-1-ol (Δ7-THC); 6a,7,8,9-tetrahydro-6,6,9-trimethyl-3-pentyl-6H-dibenzo[b,d]pyran-1-ol (Δ10-THC); (6aR,10aR)-6a,7,8,9,10,10a-hexahydro-6,6-dimethyl-9-methylene-3-pentyl-6H-dibenzo[b,d]pyran-1-ol (Δ9, 11-THC). It may or may not be possible to substitute an endocannabinoid, a THC precursor, a THC metabolite, a synthetic agonist of a cannabinoid receptor (CB₁ or CB₂ receptor, possibly specific for either CB₁ or CB₂) or an orphan receptor (GPR3, GPR6, GPR12, GPR18, or GPR55), or any combination thereof as the THC compound. If the THC compound's molecular weight is significantly different from Δ⁹-THC, the THC compound's amount may be adjusted for its bioavailability and equivalent cannabinoid receptor signaling activity as compared to Δ⁹-THC.

In one embodiment, a THC compound may or may not be administered with CBD or another non-THC cannabinoid compound, CBD and non-THC cannabinoid compounds may or may not be detectable in a composition administered, or at least they are not co-administered with the THC compound. For example, the weight or molar ratio of THC to CBD may or may not be greater than 2:1, greater than 9:1, or greater than 99:1. In another embodiment, the weight or molar ratio of THC compound to non-THC cannabinoid compounds (e.g., CBD, CBC, and CBG) may or may not be greater than 2:1, greater than 9:1, or greater than 99:1. A crude extract containing a mixture of cannabinoid compounds (e.g., nabixomols) may or may not be administered. Alternatively, less than about 1% (wt/vol) of CBD may or may not be used for treatment. In another embodiment, THC compound may be administered along with undetectable or only trace amounts (e.g., less than about 1% wt/vol) of non-THC cannabinoid compounds (e.g., CBD, CBC, and CBG). CBN may be detectable because THCA can convert to cannabinolic acid (CBNA), which can be decarboxylated to form CBN.

Hydrophobic THC compounds are poorly soluble in water and other aqueous solutions. They may be dispersed or emulsified in solvent. For example, 90% or more may or may not have a largest dimension less than 500 nm (e.g., more than 50% may have a largest dimension between about 10 nm and about 300 nm, or more than 50% may have a largest dimension between about 50 nm and about 200 nm). See Heilscher (Dan Eur Nano Sys Workshop-ENS, 2005 December, 138-143). The conventional process described therein may be used to emulsify a THC compound and other hydrophobic compounds using ultrasonic cavitation to produce a stable oil-in-water emulsion. Other conventional processes are known in the art to dissolve in solvent, disperse in a colloid or a suspension, or emulsify a THC compound (e.g., a nano-THC compound).

Melatonin is a dietary supplement, which has been associated with circadian rhythm and regulating the sleep-wake cycle. Melatonin is lipophilic and, thus, typically insoluble in water. Artificial (pure) melatonin made by chemical synthesis and purified melatonin isolated from plants (melatonin isolate) in solid form (i.e., amorphous or crystalline) would be suitable for treating a subject by administering melatonin crystals. A solid form of melatonin may be ground into a powder.

A “melatonin compound” may be one or more of, without limitation, circadin, N-acetyl-5-methoxytryptamine, a melatonin receptor agonist (e.g., agomelatine, ramelteon, tasimelteon), a melatonin precursor (e.g., 5-methoxytryptamine, N-acetyl-serotonin), or any combination thereof. It may or may not be possible to substitute a precursor in melatonin biosynthesis, a synthetic agonist of a melatonin receptor (MT₁ or MT₂ receptor, possibly MT₂), a neurotransmitter, or any combination thereof as the melatonin compound. If the melatonin compound's molecular weight is significantly different from melatonin, the melatonin compound's amount may be adjusted for its bioavailability and equivalent melatonin receptor signaling activity as compared to melatonin. Here, the melatonin compound's utility may not be a consequence of binding to its cognate receptor.

Hydrophobic melatonin compounds are poorly soluble in water and other aqueous solutions. They may be dispersed or emulsified in solvent. For example, 90% or more may or may not have a largest dimension less than 500 nm (e.g., more than 50% may have a largest dimension between about 10 nm and about 300 nm, or more than 50% may have a largest dimension between about 50 nm and about 200 nm). See Heilscher (Dan Eur Nano Sys Workshop-ENS, 2005 December, 138-143). The conventional process described therein may be used to emulsify a melatonin compound and other hydrophobic compounds using ultrasonic cavitation to produce a stable oil-in-water emulsion. Other conventional processes are known in the art to dissolve in solvent, disperse in a colloid or a suspension, or emulsify a melatonin compound (e.g., a nano-melatonin compound).

At least an anhydrous or low-moisture, THC compound and at least an anhydrous or low-moisture, melatonin compound would be suitable for manufacturing hard capsules or tablets (i.e., a plurality of unit doses). Alternatively, at least a THC compound and at least a melatonin compound may be dissolved in any of the following, without limitation, alcohols, emulsions (e.g., a nano-THC compound, a nano-melatonin compound), glycerol, micelles and lipid vesicles, microspheres, nanoparticles, oils and waxes, water and other aqueous solutions, or any combination thereof (e.g., ethanol-water). A composition having at least a THC compound and at least a melatonin compound in solid, semisolid, or liquid form could be encapsulated in soft gelatin capsules (i.e., a plurality of unit doses).

THC and melatonin compounds as described herein may be administered together at the same time or separately at different times. For example, consecutively one followed by the other back-to-back (i.e., a THC compound followed by a melatonin compound or a melatonin compound followed by a THC compound) or one immediately after waking and the other immediately before sleeping (i.e., a THC compound in the morning and a melatonin compound in the evening or a melatonin compound in the morning and a THC compound in the evening). A THC compound and a melatonin compound may be together or separate in the same or different compartments, respectively, of a kit of parts. Split doses may be needed if a THC compound and a melatonin compound are administered at different times in the day or more than once per day.

Curcumin and rutin are plant-derived flavonoids having antimicrobial activity, which may be used as preservatives in compositions. They may also be included in compositions as preservatives because of their antioxidant activity. Like melatonin, curcumin and rutin are dietary supplements. Large amounts of each are generally recognized as safe (GRAS) in food and medical products. A rich source of curcumin is turmeric, which contains several percent curcuminoids (mostly curcumin and derivatives thereof). Rutin is a quercetin glycoside linking quercetin and rutinose. Polyphenolic compounds such as curcumin and rutin are only poorly soluble in water and other aqueous solutions, but they may be dissolved in oils, alcoholic water (e.g., about 70%-95% ethanol or isopropanol in water), emulsions (e.g., nano-curcumin, nano-rutin), or lipids (e.g., micelles, vesicles). A Vitamin E-related compound may enhance the effectiveness of the polyphenolic compounds.

It may be possible to substitute other polyphenolic compounds (e.g., flavonoids) for curcumin and/or rutin. Turmeric, a curcuminoid other than curcumin, a curcumin isomer, a structural derivative of curcumin, demethoxycurcumin, bisdemethoxycurcumin, dihydro-curcumin, tetrahydrocurcumin, hexahydrocurcumin, octahydrocurcumin, or cyclocurcumin may or may not substitute for curcumin. Thus, a “curcumin-related compound” may be selected from at least curcumin, turmeric, curcuminoids, curcumin isomers, structural derivatives of curcumin, or any combination thereof. A quercetin glycoside other than rutin, a quercetrin glycoside other than rutin, a rutinoside other than rutin, a rutin isomer, a structural derivative of rutin, avicularin, didymin, dihydroxyethylrutoside, diosmin, eriocitrin, hesperidin, hyper-oside, isorhamnetin, isoquercetin, monoxerutin, narcissin, narirutin, nicotiflorin, quercetrin, spiraeoside, tetrahydroxyethylrutoside, or troxerutin may or may substitute for rutin. Thus, a “rutin-related compound” may be selected from at least rutin, quercetin glycosides, rutinosides, rutin isomers, structural derivatives of rutin, or any combination thereof.

At least a THC compound, at least a melatonin compound, at least a curcumin-related compound, at least a rutin-related compound, and at least a Vitamin E-related compound may be administered together at the same time or separately at different times. For example, consecutively one followed by the other back-to-back (e.g., a curcumin-related compound followed by a rutin-related compound, a rutin-related compound by a curcumin-related compound) or one immediately after waking and the other immediately before sleeping (e.g., a curcumin-related compound in the morning and a rutin-related compound in the evening, a rutin-related compound in the morning and a curcumin-related compound in the evening. At least a THC compound, at least a melatonin compound, at least a curcumin-related compound, at least a rutin-related compound, and at least a Vitamin E-related compound may be provided in a device or a kit of parts. For example, the compounds may be provided at the same location (e.g., reservoir) or distinct locations of a device. Alternatively, the compounds may be located together in the same compartment or separately in distinct compartments of a kit of parts. Split doses may be needed if a curcumin-related compound and a rutin-related compound are administered at different times in the day or more than once per day.

At least a THC compound, at least a melatonin compound, at least a curcumin-related compound, at least a rutin-related compound, at least a Vitamin E-related compound, and at least two or more pharmaceutically-acceptable excipients (which are anhydrous or low-moisture, or they can readily be made anhydrous or low-moisture) would be suitable for manufacturing hard capsules or tablets (i.e., a plurality of unit doses). Alternatively, at least a THC compound, at least a melatonin compound, at least a curcumin-related compound, at least a rutin-related compound, at least a Vitamin E-related compound, and at least two or more pharmaceutically-acceptable excipients may be dissolved in any of the following, without limitation, alcohols, emulsions (e.g., nanoemulsions), glycerol, micelles and lipid vesicles, microspheres, nanoparticles, oils and waxes, water and other aqueous solutions, or any combination thereof (e.g., ethanol-water). A composition comprising a THC compound, a melatonin compound, a curcumin-related compound, a rutin-related compound, a Vitamin E-related compound, and two or more pharmaceutically-acceptable excipients in solid, semisolid, or liquid form could be encapsulated in soft gelatin capsules (i.e., a plurality of unit doses).

Hydrophobic polyphenolic compounds are poorly soluble in water and other aqueous solutions. They may be dispersed or emulsified in solvent. For example, 90% or more may or may not have a largest dimension less than 500 nm (e.g., more than 50% may have a largest dimension between about 10 nm and about 300 nm, or more than 50% may have a largest dimension between about 50 nm and about 200 nm). See Heilscher (Dan Eur Nano Sys Workshop-ENS, 2005 December, 138-143). The conventional process described therein may be used to emulsify polyphenolic compounds and other hydrophobic compounds using ultrasonic cavitation to produce a stable oil-in-water emulsion. Other conventional processes are known in the art to dissolve in solvent, disperse in a colloid or a suspension, or emulsify two or more polyphenolic compounds (e.g., nano-polyphenolic compounds). A Vitamin E-related compound may enhance the solubility of the polyphenolic compounds.

At least a THC compound, at least a melatonin compound, at least two or more polyphenolic compounds (e.g., curcumin, rutin, related compounds), and at least a Vitamin E-related compound may be administered together at the same time or separately at different times. For example, they may be administered consecutively one followed by another in any order. Alternatively, any one, two, three, four, five or more compounds may be administered immediately after waking and the remaining compounds immediately before sleeping. At least a THC compound, at least a melatonin compound, at least two or more polyphenolic compounds (e.g., curcumin, rutin, related compounds), and at least a Vitamin E-related compound may be together or separate in the same or different compartments, respectively, of a kit of parts. Split doses may be needed if any one, two, three, four, five or more compounds are administered at different times in the day or more than once per day.

At least a THC compound, at least a melatonin compound, at least two or more polyphenolic compounds (e.g., curcumin, rutin, related compounds), at least a Vitamin E-related compound, and at least two or more pharmaceutically-acceptable excipients (all of which are anhydrous or low-moisture, or they can readily be made anhydrous or low-moisture) would be suitable for manufacturing hard capsules or tablets (i.e., a plurality of unit doses). Alternatively, at least a THC compound, at least a melatonin compound, at least two or more polyphenolic compounds (e.g., curcumin, rutin, related compounds), at least a Vitamin E-related compound (e.g., alpha-tocopherol), and at least two or more pharmaceutically-acceptable excipients may be dissolved in any of the following, without limitation, alcohols, emulsions (e.g., nanoemulsions), glycerol, micelles and lipid vesicles, microspheres, nanoparticles, oils and waxes, water and other aqueous solutions, or any combination thereof (e.g., ethanol-water). A composition having polyphenolic compounds in solid, semisolid, or liquid form could be encapsulated in a soft gelatin capsule. A composition comprising a THC compound, a melatonin compound, two or more polyphenolic compounds (e.g., curcumin, rutin, related compounds), a Vitamin E-related compound, and two or more pharmaceutically-acceptable excipients in solid, semisolid, or liquid form could be encapsulated in soft gelatin capsules (i.e., a plurality of unit doses).

Carriers of the composition may be at least an emulsion (e.g., nanoemulsions), polymeric micelles, lipid vesicles, solid or porous particles (e.g., nanoparticles), or any combination thereof. THC, melatonin, curcumin-related, rutin-related, and Vitamin E-related compounds may all be associated with the same group of carriers. For example, particulate carriers may be associated with any one, two, three, four, five, or more compounds. Alternatively, one or more of THC, melatonin, curcumin-related, rutin-related, or Vitamin E-related compound may be associated with a different carrier. For example, particulate carriers associated any compound may be associated with one and only one compound. All compounds may be mixed and the mixture incorporated or encapsulated in carriers; each compound may be incorporated or encapsulated in carriers separately, then the different carriers are mixed together. Stable emulsions or lipid vesicles may be manufactured by processes such as ultra-high speed shearing and ultrasonic cavitation. After dissolving one or more hydrophobic compounds in solvent (e.g., an oil), nanoparticles may be produced therefrom: e.g., nanolipo-somes having liquid cores and phospholipid bilayer membranes, nanolipospheres having hydrophobic solid cores and phospholipid monolayer membranes, nanoemulsions having liquid lipid cores and phospholipid monolayer membranes, solid lipid nanoparticles having solid lipid cores and phospholipid monolayer membranes, nanostructured lipid carriers having liquid/solid lipid cores and phospholipid monolayer membranes, polymeric nanoparticles having hydrophilic polymer shells, and metallic nanoparticles having surface coated with a nanoemulsion. See Vegallo (Nanomaterials. 2020 June, 10: 1232); which is incorporated by reference herein.

Examples of emulsifiers include, but are not limited to, cyclodextrins, fatty alcohols, lecithin or phospholipids, poloxamers, polyethylene glycols (e.g., PEG 1000), polyglycerol fatty acid esters, polysorbates (e.g., polysorbate 80 or Tween 80), saponins, and sorbitan fatty acid esters (e.g., sorbitan oleate or Span 80). Surfactants are often emulsifiers; emulsifiers and surfactants may also be considered solubilizers.

Vehicles of the composition may be aqueous or nonaqueous solutions or mixtures of two or more solvents (e.g., H₂O+DMSO). Water, saline, buffered (possibly hypotonic or isotonic) aqueous solutions, and dimethyl sulfoxide may be used as solvents for a water-soluble (preferably hydrophilic) compound. Alcohols (possibly diluted to a final concentration for administration of about 5%-20% or about 10%-15% ethanol in water), N,N-dimethylacetamide, glycerol, hydrogenated vegetable oils, mineral oil, vegetable oils, PEG 1000, polysorbate 80, propylene glycol, medium chain triglycerides (MCTs may be caprylic acid, capric acid, or both), and dimethyl sulfoxide may be used as a solvent for one or more hydrophobic compounds.

### Compositions (Medicaments, Medical Devices, and Kits of Parts)

Compositions may comprise (i) a THC compound and (ii) a melatonin compound (or a salt of either or both compounds, or a solvate such as a hydrate of either or both compounds, or an analog of either or both compounds sharing the same or similar therapeutic properties, or a structural derivative of either or both compounds having the same or similar therapeutic effects, or a stereoisomer of either or both compounds, or a polymorph of either or both compounds, or a metabolite of either or both compounds, or a prodrug of either or both compounds) as described herein, (iii) optional polyphenolic compounds (e.g., curcumin and rutin, compounds related thereto) as described herein, (iv) optional Vitamin E-related compound (e.g., alpha-tocopherol) as described herein, (v) optional at least one chemotherapeutic agent (see the exemplary agents listed below), (vi) a plurality of pharmaceutically-acceptable excipients, (vii) optional pharmaceutically-acceptable vehicle, and (viii) optional pharmaceutically-acceptable carrier.

Compositions may comprise therapeutically effective amounts of (i) a THC compound and (ii) a melatonin compound (or a salt of either or both compounds, or a solvate such as a hydrate of either or both compounds, or an analog of either or both compounds sharing the same or similar therapeutic properties, or a structural derivative of either or both compounds having the same or similar therapeutic effects, or a stereoisomer of either or both compounds, or a polymorph of either or both compounds, or a metabolite of either or both compounds, or a prodrug of either or both compounds) as described herein, (iii) optional effective amounts of polyphenolic compounds (e.g., curcumin and rutin, compounds related thereto) as described herein, (iv) optional effective amount of a Vitamin E-related compound (e.g., alpha-tocopherol) as described herein, (v) optional effective amount of at least one chemotherapeutic agent (the next two paragraphs list exemplary agents), (vi) a plurality of pharmaceutically-acceptable excipients, (vii) optional pharmaceutically-acceptable vehicle, and (viii) optional pharmaceutically-acceptable carrier.

In some embodiments, at least one chemotherapeutic agent can be combined with at least two compounds as described herein (i.e., a THC compound and a melatonin compound) or at least five compounds as described herein (i.e., additionally polyphenolic compounds such as curcumin and rutin, Vitamin E, and compounds related thereto) for treating a neuropsychiatric symptom or a neurologic disorder. A chemotherapeutic agent may be selected from, without limitation, antidepressants, antipsychotics, mood stabilizers, selective serotonin reuptake inhibitors, serotonin/norepinephrine reuptake inhibitors, and anxiolytics. For example, the chemotherapeutic agent may be a selective serotonin reuptake inhibitor (e.g., citalopram, escitalopram, fluvoxamine, sertaline, vortioxetine); a serotonin/norepinephrine reuptake inhibitor (e.g., desvenlafaxine, duloxetine, levomilnacipran, venlafaxine); an anti-depressant (e.g., mirtazapine, trazodone); a mood stabilizer (e.g., carbamazepine, gabapentin, lamotrigine, oxcarbazepine topiramate, valproic acid); an antipsychotic (e.g., aripiprazole, haloperidol, olanzapine, perphenazine, quetiapine, risperidone, ziprasidone); an anxiolytic (e.g., buspirone, gepirone, ipsapirone, tandospirone); and a orexin receptor antagonist (e.g., lemborexant, suvorexant). The chemotherapeutic agent may be co-administered with the THC and melatonin compounds as described herein (the at least two, three, four, five, or more compounds together in the same composition once and only once per day, synchronously in identical divided compositions more than once per day, separately in their own composition once and only once per day, or separately in their own composition at two different times per day.

In other embodiments, at least one chemotherapeutic agent can be combined with at least two compounds as described herein (i.e., a THC compound and a melatonin compound) or at least five compounds as described herein (i.e., additionally polyphenolic compounds such as curcumin and rutin, Vitamin E, and compounds related thereto) for treating dementia or Alzheimer's disease. A chemotherapeutic agent may be selected from, without limitation, acetylcholinesterase inhibitors, antibodies binding beta-amyloid, antibodies binding tau protein, beta-amyloid aggregation inhibitors, beta-secretase inhibitors, gamma-secretase inhibitors, kinase inhibitors, receptor antagonists, tau phosphorylation inhibitors, and tau protein aggregation inhibitors. The chemotherapeutic agent may be aducanumab, amantadine, bepranemab, brexpiprazole, bryostatin, colostrinin, donanemab, donepezil, Ginko biloba extract EGb 761, galantamine, gantesnerumab, gosuranemab, lanabecestat, lecanemab, leucomethylthioninium, levetiracetam, memantine, methylphenidate, neflamapimod, prasinezumab, rivastigmine, semorinemab, solanezumab, tilavonemab, tramiprosate, tricaprilin, zagotenemab, or any combination thereof. The chemotherapeutic agent may be co-administered with the at least two, three, four, five, or more compounds together in the same composition once and only once per day, synchronously in identical divided compositions more than once per day, separately in their own composition once and only once per day, or separately in their own composition at two different times per day.

Medicaments are pharmaceutically acceptable and may comprise any of the compositions described herein. A medicament may be any of the following, without limitation: aerosols, capsules, chartulae and sachets, chewable candies and gums, colloids, disintegrating strips and tabs, drops, elixirs, foams and mousses, hydrogels, lozenges and pastilles, mouthwashes and oral rinses, solutions, suspensions, tablets, and tonics; the contents of a sealed container such as, without limitation, an ampule, a blister pack (i.e., blister card or blister pouch), a pill bottle, a pillbox, or a vial; or the contents of a device such as, without limitation, an inhaler, an insufflator, a mister or a sprayer, a nebulizer, a suppository, a syringe, or a vaporizer. A use for the medicament may be administering THC and melatonin compounds as described herein, optional polyphenolic compounds (e.g., curcumin and rutin, compounds related thereto) as described herein, optional at least one chemotherapeutic agent as described herein, optional pharmaceutically-acceptable excipients, optional pharmaceutically-acceptable vehicle, and optional pharmaceutically-acceptable carrier by any route such as, but not limited to, at least enteral administration, parenteral administration, mucosal administration, pulmonary administration, or any combination thereof.

At least one chemotherapeutic agent may be administered together with two or more compounds as described herein (e.g., THC compounds; melatonin compounds; optional polyphenolic compounds such as curcumin and rutin, optional Vitamin E, and compounds related thereto) or separately therefrom. At least one chemotherapeutic agent may be administered enterally (i.e., digested and adsorbed in the gastrointestinal tract, likely peroral) or parenterally (i.e., all other routes) for a systemic effect after entering the circulation. Enteral administration of at least one chemotherapeutic agent may use a capsule, a chewable candy or gum, a disintegrating strip or tab, an elixir, a lozenge or pastille, a tablet, or a tincture. Parenteral administration of at least one chemotherapeutic agent may use a hypodermic syringe (e.g., intramuscular, intravenous, subcutaneous), an implanted depot, an implanted pump, an infusion pump, or an intravenous drip through a cannula/catheter.

Pulmonary administration (e.g., inhalation, insufflation) of at least one chemotherapeutic drug may achieve a local effect in the lungs or a systemic effect by adsorption though alveolar epithelium directly into the circulation, also bypassing first-pass hepatic metabolism. Examples of other routes of administration include, but are not limited to, the ears for otic drops, the eyes for optic drops, the nose for nasal drops and sprays, the rectum for rectal enemas and suppositories, and the vagina for vaginal enemas and suppositories.

Excipients of particular interest may have properties desirable for formulating compositions and manufacturing products: e.g., preservatives (e.g., antimicrobials, antioxidants, stabilizers), emulsifiers, solubilizers, surfactants, and other food ingredients generally recognized as safe (GRAS). Examples of chemicals that may be suitable for use in the compositions and products as pharmaceutically-acceptable excipients are, without limitation, one or more acetates, alcohols (e.g., benzyl alcohol, ethanol, isopropanol), aluminum silicate, aluminum stearate, amino acids (e.g., arginine, aspartic acid, glutamic acid, glycine, histidine, methionine), ascorbic acid (Vitamin C), beta-carotene, carbomers, dendrimers, fatty acid esters (e.g., cetyl palmitate, methyl linoleate, isopropyl myristate, isopropyl palmitate), fatty alcohols (e.g., cetyl alcohol, octyldodecanol, oleyl alcohol, stearyl alcohol), free fatty acids (e.g., caprylic acid, capric acid, myristic acid, oleic acid, palmitic acid, stearic acid), gelatin, hydrogenated vegetable oils, magnesium silicate, magnesium stearate, medium chain triglycerides (MCT), nonreducing sugars (e.g., raffinose, sucrose, trehalose), phospholipids (e.g., phosphatidic acid, phosphatidylcholine, phosphatidylethanolamine, phosphatidylglycerol, phosphatidylserine), polyacrylates, polyethylene glycols, polyethylene oxides, polyglycerol esters, poly(lactic-co-glycolic acid), polymethacrylates, poloxamers, polypropylene glycols, polysorbates, polyvinyl alcohols, polyvinyl polypyrrolidones, polyvinyl pyrrolidones, propylene glycol, reducing sugars (e.g., fructose, galactose, lactose, maltose), retinal (Vitamin A), saccharides (e.g., agar, alginates, alginic acid, carboxymethyl cellulose, carrageenan, cellulose acetate, chitosan, chondroitin sulfate, cyclodextrins, dextrans, dextrins, ethylcellulose, guar gum, gum tragacanth, hyaluronic acid, hydroxyethyl cellulose, hydroxypropyl methylcellulose, maltodextrins, microcrystalline cellulose, powdered cellulose, pregelatinized starches, sodium hyaluronate, starches, xanthan gum), salts (e.g., any combination of cation Ca²⁺, K⁺, Mg²⁺, Na⁺, or NH₄⁺ and anion C₂H₃O₂⁻, HCO₃⁻, Cl⁻, NO₂⁻, or PO₄³⁻), saponins, silicone oils, sodium benzoate, sugar alcohols (e.g., erythritol, glycerol, isomalt, lactitol, maltitol, mannitol, sorbitol, xylitol), syrups (e.g., agave, corn, date, honey, malt extract, molasses), titanium dioxide, tocopherols, vegetable oils, white petrolatum, zinc stearate, or any combination thereof. Note chemicals suitable for use as pharmaceutically-acceptable excipients may have multiple (possibly overlapping) properties desirable for formulating compositions and manufacturing products. See Sheskey et al. (Handbook of Pharmaceutical Excipients, Ninth Ed, 2020 October, Pharmaceutical Press); which is incorporated by reference herein for its listed excipients and their descriptions.

In some embodiments, the composition may further comprise one or more vehicles such as, aqueous solvents, nonaqueous solvents, and mixtures therebetween (e.g., alcoholic waters, alcohols, buffered and preferably hypotonic or isotonic solutions, N,N-dimethylacetamide, dimethyl sulfoxide, fats, glycerol, mineral oil, plant butters, saline, silicone oils, vegetable oils, white petrolatum, water) and/or one or more types of carriers (e.g., emulsions, micelles, particles, vesicles). For example, THC and melatonin compounds as described herein may be formulated within at least polymeric micelles, nanoparticles, an oil-in-water nanoemulsion, or lipid vesicles to improve at least the THC and melatonin compounds' solubility, stability, bioavailability, or any combination thereof.

Sterile compositions may be prepared by dissolving the compounds as described herein in effective amounts in a suitable solvent with other vehicles, carriers, and/or excipients as appropriate, followed by filter sterilization. Alternatively, solutions may be prepared by dissolving sterile compounds as described herein in an aseptic vehicle that is a dissolution medium. Colloids may be prepared by dispersing sterile compounds as described herein in an aseptic vehicle that is a colloidal medium. Suspensions may be prepared by dispersing sterile compounds as described herein in an aseptic vehicle that is a suspension medium. For compounds in solid form used to prepare a sterile infusible or injectable solution, drying by evaporation or lyophilization can yield a solid form (e.g., granules) comprising compounds as described herein along with any excipients, which may be reconstituted with a sterile vehicle-all under aseptic conditions. Systemic effects may be achievable thereby.

A “finished” composition is formulated in its solid, semisolid, or liquid form prior to being transported to the place where a subject is treated. The finished composition is preferably compatible with the chosen mode and route of administration, stable at substantially physiologic levels of pH and osmolality, and confirmed to be free of detectable pathogens and pyrogen.

### Formulations

A composition may be in any form selected from, without limitation, at any aerosol, capsule, chartula or sachet, chewable candy or gum, colloid, cream, disintegrating strip or tab, drops, elixir, foam or mousse, hydrogel, lozenge or pastille, mouthwash or oral rinse, ointment, suspension, tablet, or tincture; administered by a route selected from, without limitation, at least oral, enteral, mucosal (e.g., buccal, sublabial, sublingual), or pulmonary (i.e., inhalation, insufflation, instillation); or delivered by a dispenser selected from, without limitation, any dressing or patch, dropper or pipet, enema bottle, implanted depot or pump, infusion pump, inhaler, insufflator, intravenous (IV) bag or bottle, mister or sprayer, nebulizer, pill bottle, pillbox, suppository, syringe, or vaporizer. A systemic or local effect may be achievable thereby.

Examples of a pharmaceutically-acceptable vehicle include, but are not limited to, aqueous solutions (e.g., saline, water for injection), alcohols (e.g., ethanol), glycerol, polyethylene glycols, polypropylene glycols, propylene glycol, vegetable oils, and mixtures thereof (e.g., alcoholic water). Examples of pharmaceutically-acceptable carriers include, but are not limited to, cyclodextrins, dendrimers, lipid vesicles, microspheres, nanoparticles, nano-fibers, polymeric micelles, and stable emulsions. Note that some vehicles and some carriers may also be considered excipients.

Compositions may further comprise at least two, three, four, or more pharmaceutically-acceptable excipients including, but not limited to, preservatives (e.g., ascorbic acid, ethanol, glycerol, sodium benzoate), emulsifiers (e.g., glycerol, polysorbate 80, PEG 1000), binders, diluents or fillers, disintegrants, glidants or lubricants, buffering agents (e.g., sodium citrate), and tonicity agents (e.g., dextrose, potassium salts, sodium salts). Note that some excipients may have multiple, possibly overlapping, functions such as, for example, an emulsifier acting as a surfactant and a solublizer.

Methods of formulating a composition may comprise one or more steps of bringing into association (i.e., direct or indirect contact among different compounds) two or more compounds as described herein (e.g., THC and melatonin compounds; optional polyphenolic compounds such as curcumin and rutin, optional Vitamin E, and compounds related thereto; optional at least one chemotherapeutic agent) and one or more pharmaceutically-acceptable excipients. In some embodiments, compositions can be formulated by one or more steps of bringing into association therapeutically effective amounts of the THC and melatonin compounds as described herein; optionally, effective amounts of two or more polyphenolic compounds; optionally, an effective amount of a Vitamin E-related compound; one or more pharmaceutically-acceptable excipients; optionally, at least a vehicle and/or a carrier. A liquid form may be aseptically dispensed into a sterile container with or without an intervening step of drying the composition by conventional processes (e.g., evaporation, lyophilization) as known in the art. Alternatively, a solid form may be aseptically dispensed into a sterile container with or without an intervening step of forming the composition into a plurality of unit doses.

As described in the last paragraph, compounds in liquid form can be dried or, alternatively, compounds in solid form can be combined to provide an anhydrous composition. In other embodiments, a composition may be made anhydrous if substantial contact with moisture and/or humidity during at least manufacture, finishing, storage, or a combination thereof is expected or possible. Thus, an anhydrous composition may be formulated from low-moisture compounds and low-humidity conditions, then gently heated under vacuum to ensure any residual water and volatiles are driven off. The anhydrous composition may be formulated, transported, and stored such that its anhydrous nature is maintained. Similarly, the composition may be “finished” using barrier material to prevent exposure to air and moisture. If the anhydrous composition is in solid form, the composition may be administered in solid form or, after reconstitution with a sterile vehicle under aseptic conditions, in liquid form. Ampules, blister cards, chartulae or sachets, pill bottles, pillboxes, and vials may hold the anhydrous composition.

Water can be added to an anhydrous formulation as a means for simulating long-term storage and thereby test its stability, effectiveness after transportation and/or storage, and shelf life. Contact with oxygen and oxygenation may be avoided by blanketing or purging with inert gas during filling and other processes. Lot testing may be performed to confirm the absence of viruses, pyrogen, or other contaminants.

An anhydrous composition may comprise two or more compounds as described herein (e.g., THC and melatonin compounds; optional polyphenolic compounds such as curcumin and rutin, optional Vitamin E, and compounds related thereto; optional at least one chemotherapeutic agent). The anhydrous composition may be held in a sealed container such as, without limitation, an ampule or a vial, an intravenous (IV) bag or bottle, or the like that may be reconstituted with a sterile vehicle under aseptic conditions, then delivered with a gravity drip, a pump, or a syringe.

Processes for formulating medicaments or, by further processing, manufacturing down-stream products containing unit doses in solid form including, but not limited to, a blister card, a chartula or sachet, a chewable candy or gum, a coated or uncoated tablet, a disintegrating strip or tab, a hard or soft capsule, or a lozenge or pastille.

Capsules or tablets may be manufactured in conventional machines as known in the art. Capsules may be hard or soft. Capsules and tablets may be coated or uncoated. The coating may comprise at least a resinous or polymeric material and control release of THC and melatonin compounds as described herein. Compositions for enteral administration may be provided as enteric-coated capsules or tablets comprising THC and melatonin compounds as described herein mixed with a diluent or filler (e.g., calcium carbonate) or as enteric-coated capsules comprising THC and melatonin compounds as described herein mixed with a vehicle (e.g., alcohol, alcoholic water, oil, water). The coating on the capsule or tablet may chiefly determine their release characteristics and bioavailability; preferably, the capsule or tablet has an enteric coating for delivery of THC and melatonin compounds as described herein bypassing the stomach (and its digestive juices) and targeting the proximal end of the small intestine (e.g., duodenum) where THC and melatonin compounds as described herein would be absorbed.

### Dosages

Two or more compounds as described herein (e.g., THC compounds; melatonin compounds; optional polyphenolic compounds such as curcumin and rutin, optional Vitamin E, and compounds related thereto; optional at least one chemotherapeutic agent) can be delivered in the form of a composition comprising amounts of each compound, two or more pharmaceutically-acceptable excipients, and optional pharmaceutically-acceptable vehicle and/or carrier.

THC and melatonin compounds as described herein can be delivered in the form of compositions comprising therapeutically effective amounts of THC and melatonin compounds, optional effective amounts of two or more polyphenolic compounds (e.g., curcumin and rutin, compounds related thereto), optional effective amount of Vitamin E-related compound, and optional effective amount of at least one chemotherapeutic agent; formulated together with one or more pharmaceutically-acceptable vehicles, carriers, and/or excipients.

In some embodiments, THC and melatonin compounds as described herein, polyphenolic compounds, Vitamin E-related compound, and at least one chemotherapeutic agent are administered in separate compositions and, because of the compounds' different physical and/or chemical characteristics, can be administered by different modes and/or routes (e.g., one compound is administered enterally, while another compound is administered parenterally). In other embodiments, THC and melatonin compounds as described herein, polyphenolic compounds, and at least one chemotherapeutic agent can be administered separately, but via the same route (e.g., both enterally, both by inhalation, both parenterally). For convenience, it is expected that THC and melatonin compounds as described herein, two or more polyphenolic compounds, and at least one chemotherapeutic agent will be administered in the same composition by a peroral and/or nasal route.

The selected dosage level will depend upon a variety of factors including, for example, the activity of the particular compound, the route of administration, the time of administration, the rate of excretion or metabolism of the particular compound, the rate and extent of its absorption, the duration of treatment, other compounds and/or materials used in combination with the particular compound, the age, sex, weight, condition, general health and prior medical history of the subject being treated, and like factors well known in the medical arts.

A suitable amount of a tetrahydrocannabinol (THC) compound may be from about 2.0 mg to about 7.0 mg per day, or the amount may be a daily dosage from about 0.0286 mg/kg to about 0.1000 mg/kg of a subject's bodyweight. A suitable amount of a THC compound may be from about 2.25 mg to about 6.75 mg per day, or the amount may be a daily dosage from about 0.0321 mg/kg to about 0.0964 mg/kg of a subject's bodyweight. A suitable amount of a THC compound may be from about 2.5 mg to about 6.5 mg per day, or the amount may be a daily dosage from about 0.0357 mg/kg to about 0.0929 mg/kg of a subject's bodyweight. A suitable amount of a THC compound may be from about 2.75 mg to about 6.25 mg per day, or the amount may be a daily dosage from about 0.0393 mg/kg to about 0.0893 mg/kg of a subject's bodyweight. A suitable amount of a THC compound may be from about 3.0 mg to about 6.0 mg per day, or the amount may be a daily dosage from about 0.0429 mg/kg to about 0.0857 mg/kg of a subject's bodyweight.

A suitable amount of a melatonin compound may be from about 1.0 mg to about 4.4 mg per day, or the amount may be a daily dosage from about 0.0143 mg/kg to about 0.0629 mg/kg of a subject's bodyweight. A suitable amount of a melatonin compound may be from about 1.6 mg to about 4.4 mg per day, or the amount may be a daily dosage from about 0.0229 mg/kg to about 0.0629 mg/kg of a subject's bodyweight. A suitable amount of a melatonin compound may be from about 1.25 mg to about 4.2 mg per day, or the amount may be a daily dosage from about 0.0179 mg/kg to about 0.0600 mg/kg of a subject's bodyweight. A suitable amount of a melatonin compound may be from about 1.6 mg to about 4.2 mg per day, or the amount may be a daily dosage from about 0.0229 mg/kg to about 0.0600 mg/kg of a subject's bodyweight. A suitable amount of a melatonin compound may be from about 1.0 mg to about 4.2 mg per day, or the amount may be a daily dosage from about 0.0143 mg/kg to about 0.0600 mg/kg of a subject's bodyweight. A suitable amount of a melatonin compound may be from about 1.25 mg to about 4.0 mg per day, or the amount may be a daily dosage from about 0.0179 mg/kg to about 0.0571 mg/kg of a subject's bodyweight. A suitable amount of a melatonin compound may be from about 1.6 mg to about 4.0 mg per day, or the amount may be a daily dosage from about 0.0229 mg/kg to about 0.0571 mg/kg of a subject's bodyweight. A suitable amount of a melatonin compound may be from about 1.5 mg to about 4.0 mg per day, or the amount may be a daily dosage from about 0.0215 mg/kg to about 0.0571 mg/kg of a subject's bodyweight. A suitable amount of a melatonin compound may be from about 1.75 mg to about 3.75 mg per day, or the amount may be a daily dosage from about 0.0250 mg/kg to about 0.0536 mg/kg of a subject's bodyweight. A suitable amount of a melatonin compound may be from about 1.75 mg to about 4.0 mg per day, or the amount may be a daily dosage from about 0.0250 mg/kg to about 0.0571 mg/kg of a subject's bodyweight. A suitable amount of a melatonin compound may be from about 2.0 mg to about 3.5 mg per day, or the amount may be a daily dosage from about 0.0286 mg/kg to about 0.0500 mg/kg of a subject's bodyweight.

A suitable amount of a curcumin-related compound may be from about 0.3 mg to about 1.5 mg per day, or the amount may be a daily dosage from about 0.0043 mg/kg to about 0.0215 mg/kg of a subject's bodyweight. A suitable amount of a curcumin-related compound may be from about 0.3 mg to about 1.2 mg per day, or the amount may be a daily dosage from about 0.0043 mg/kg to about 0.0171 mg/kg of a subject's bodyweight. A suitable amount of a curcumin-related compound may be from about 0.6 mg to about 1.2 mg per day, or the amount may be a daily dosage from about 0.0086 mg/kg to about 0.0171 mg/kg of a subject's bodyweight. A suitable amount of a curcumin-related compound may be from about 0.6 mg to about 1.5 mg per day, or the amount may be a daily dosage from about 0.0086 mg/kg to about 0.0215 mg/kg of a subject's bodyweight.

A suitable amount of a rutin-related compound may be from about 0.3 mg to about 1.5 mg per day, or the amount may be a daily dosage from about 0.0043 mg/kg to about 0.0215 mg/kg of a subject's bodyweight. A suitable amount of a rutin-related compound may be from about 0.3 mg to about 1.2 mg per day, or the amount may be a daily dosage from about 0.0043 mg/kg to about 0.0171 mg/kg of a subject's bodyweight. A suitable amount of a rutin-related compound may be from about 0.6 mg to about 1.2 mg per day, or the amount may be a daily dosage from about 0.0086 mg/kg to about 0.0171 mg/kg of a subject's bodyweight. A suitable amount of a rutin-related compound may be from about 0.6 mg to about 1.5 mg per day, or the amount may be a daily dosage from about 0.0086 mg/kg to about 0.0215 mg/kg of a subject's bodyweight.

A suitable amount of two or more polyphenolic compounds may be from about 0.1 mg to about 5.0 mg per day, or the amount may be a daily dosage from about 0.0014 mg/kg to about 0.0714 mg/kg of a subject's bodyweight. A suitable amount of two or more polyphenolic compounds may be from about 0.3 mg to about 5.0 mg per day, or the amount may be a daily dosage from about 0.0043 mg/kg to about 0.0714 mg/kg of a subject's bodyweight. A suitable amount of two or more polyphenolic compounds may be from about 0.1 mg to about 4.0 mg per day, or the amount may be a daily dosage from about 0.0014 mg/kg to about 0.0571 mg/kg of a subject's bodyweight. A suitable amount of two or more polyphenolic compounds may be from about 0.3 mg to about 4.0 mg per day, or the amount may be a daily dosage from about 0.0043 mg/kg to about 0.0571 mg/kg of a subject's bodyweight. A suitable amount of two or more polyphenolic compounds may be from about 0.3 mg to about 3.0 mg per day, or the amount may be a daily dosage from about 0.0043 mg/kg to about 0.0429 mg/kg of a subject's bodyweight. A suitable amount of two or more polyphenolic compounds may be from about 0.6 mg to about 5.0 mg per day, or the amount may be a daily dosage from about 0.0086 mg/kg to about 0.0714 mg/kg of a subject's bodyweight. A suitable amount of two or more polyphenolic compounds may be from about 0.6 mg to about 4.0 mg per day, or the amount may be a daily dosage from about 0.0086 mg/kg to about 0.0571 mg/kg of a subject's bodyweight. A suitable amount of two or more polyphenolic compounds may be from about 0.6 mg to about 3.0 mg per day, or the amount may be a daily dosage from about 0.0086 mg/kg to about 0.0429 mg/kg of a subject's bodyweight. A suitable amount of two or more polyphenolic compounds may be from about 0.6 mg to about 2.4 mg per day, or the amount may be a daily dosage from about 0.0086 mg/kg to about 0.0343 mg/kg of a subject's bodyweight. A suitable amount of two or more polyphenolic compounds may be from about 0.9 mg to about 5.0 mg per day, or the amount may be a daily dosage from about 0.0129 mg/kg to about 0.0714 mg/kg of a subject's bodyweight. A suitable amount of two or more polyphenolic compounds may be from about 0.9 mg to about 4.0 mg per day, or the amount may be a daily dosage from about 0.0129 mg/kg to about 0.0571 mg/kg of a subject's bodyweight. A suitable amount of two or more polyphenolic compounds may be from about 0.9 mg to about 3.0 mg per day, or the amount may be a daily dosage from about 0.0129 mg/kg to about 0.0429 mg/kg of a subject's bodyweight. A suitable amount of two or more polyphenolic compounds may be from about 1.2 mg to about 2.4 mg per day, or the amount may be a daily dosage from about 0.0171 mg/kg to about 0.0343 mg/kg of a subject's bodyweight. A suitable amount of two or more polyphenolic compounds may be from about 1.2 mg to about 3.0 mg per day, or the amount may be a daily dosage from about 0.0171 mg/kg to about 0.0429 mg/kg of a subject's bodyweight. The polyphenolic compounds may be one and only one curcumin-related compound and one and only one rutin-related compound in approximately the same amounts (e.g., 1.1:1, 1.2:1, 1.5:1, 1.7:1, 1.8:1, 1:1, 1:1.8, 1:1.7, 1:1.5, 1:1.2,1:1.1, 1-1.8:1-1.8 or from 1.8:1 to 1:1.8, from 1.9:1 to 1:1.9, from 1.7:1 to 1:1.7, from 1.5:1 to 1:1.5, from 1.2:1 to 1:1.2, from 1.1:1 to 1:1.1).

Any suitable amount of a Vitamin E-related compound may be equivalent to an amount of alpha-tocopherol from about 20 mg to about 55 mg per day, or the amount may be a daily dosage equivalent to an amount of alpha-tocopherol from about 0.286 mg/kg to about 0.786 mg/kg of a subject's bodyweight. A suitable amount of a Vitamin E-related compound may be equivalent to an amount of alpha-tocopherol from about 30 mg to about 45 mg per day, or the amount may be a daily dosage equivalent to an amount of alpha-tocopherol from about 0.429 mg/kg to about 0.643 mg/kg of a subject's bodyweight. A suitable amount of a Vitamin E-related compound may be equivalent to an amount of alpha-tocopherol from about 35 mg to about 40 mg per day, or the amount may be a daily dosage equivalent to an amount of alpha-tocopherol from about 0.500 mg/kg to about 0.571 mg/kg of a subject's bodyweight.

Effective amounts of the compounds as described herein (e.g., THC compounds; melatonin compounds; optional polyphenolic compounds such as curcumin and rutin, and compounds related thereto; optional at least one chemotherapeutic agent) can be varied to obtain amounts of the compounds that are effective to achieve the desired therapeutic response for a particular subject and mode of administration without being toxic or otherwise harmful. In some embodiments, a dosage less than the lower limit of the aforesaid ranges is sufficient to be therapeutically effective, although a dosage more than their upper limit might be administered without adverse effect by, for example, dividing a large dosage into many small doses for more frequent administration within a time period.

The compounds as described herein (e.g., THC compounds; melatonin compounds; optional polyphenolic compounds such as curcumin and rutin, and compounds related thereto; optional at least one chemotherapeutic agent) may be administered as one or more compositions enterally (digestion and adsorption in the gastrointestinal tract, likely peroral) or parenterally (all other routes) for a systemic effect after entering the circulation. In particular, a capsule, a chewable candy or gum, a disintegrating strip or tab, an elixir, a tablet, or a tincture may be enterally administered. Alternatively, a hypodermic syringe (e.g., intramuscular, intravenous, subcutaneous), an implanted depot or pump, an infusion pump, an intravenous (IV) drip, or the like may be used for parenteral administration.

In general, a suitable amount of a chemotherapeutic agent will be the lowest amount effective to produce the desired therapeutic effect. Suitable amounts will generally depend upon factors such as those discussed above. A daily dosage of a chemotherapeutic agent for a 70-kg subject may be from about 0.0001 mg to about 1000 mg, or from about 0.01 mg to about 1000 mg, or from about 1 mg to about 1000 mg, or from about 0.001 mg to about 500 mg, or from about 0.01 mg to about 500 mg, or from about 0.1 mg to about 500 mg, or from about 1 mg to about 500 mg, or from about 0.01 mg to about 100 mg, or from about 0.1 mg to about 100 mg, or from about 1 mg to about 100 mg, or from about 0.001 mg to about 50 mg, or from about 0.01 mg to about 50 mg, or from about 0.1 mg to about 50 mg, or from about 1 mg to about 50 mg, or from about 0.001 mg to about 10 mg, or from about 0.01 mg to about 10 mg, or from about 0.1 mg to about 10 mg, or from about 1 mg to about 10 mg.

The chemotherapeutic agent may be administered daily (preferably once and only once), every other day, every week, biweekly, or every month. The dosing schedule can include a “drug holiday,” e.g., the chemotherapeutic agent can be administered for two weeks on, one week off, or three weeks on, one week off, or four weeks on, one week off, etc., or continuously, without a drug holiday. In some embodiments, the therapeutically effective amount of the chemotherapeutic agent may decrease if administered in combination therapy.

The chemotherapeutic agent is administered in a single unit dose or a plurality of unit doses within a time period. Generally, a dose is the daily amount to be at least given to or taken by a subject. For example, once, twice, three times, four times, or six times per day. Dosing can be about once a month, about once every two weeks, about once a week, or about once every other day. The chemotherapeutic agent may be co-administered, along with along with THC and melatonin compounds as described herein, at a frequency ranging from once per day (daily) to six times per day. In other embodiments, they can be co-administered synchronously for less than about 7 days, more than about 6 days, less than about 10 days, more than about two weeks, less than about 15 days, more than about three weeks, less than about 30 days, more than about one month, less than about 60 days, more than about two months, less than about 90 days, more than about three months, less than about 120 days, more than about six months, less than about 360 days, or more than about one year. In some cases, dosing is continuous for as long as necessary to achieve a beneficial effect. In other cases, dosing is reduced after achieving a therapeutically effective amount systemically or a therapeutic benefit.

Administration of compositions as provided herein can continue as long as necessary. In some embodiments, the chemotherapeutic agent is administered for more than about one day, more than about one week, more than about two weeks, more than about three weeks, more than about one month, more than about two months, more than about three months, more than about six months, or more than about one year. In some embodiments, compositions are administered for less than about 28 days, less than 30 days, less than about 60 days, a less than bout 120 days, or less than about 360 days. In some embodiments, the chemotherapeutic agent is administered for about one day, about one week, about two weeks, about one month, about two months, about three months, about six months, or about one year. In some embodiments, the chemotherapeutic agent is administered chronically on an ongoing basis, e.g., for treatment of chronic effects.

Since THC and melatonin compounds as described herein can be administered in combination with other treatments (e.g., psychological counseling, psychotherapy, surgery, synchronously administering an additional chemotherapeutic agent), the dose of each compound or the frequency of treatment can be lower than the corresponding dose for single-agent therapy. Alternatively, the daily dosage for the chemotherapeutic agent may be an amount from about 0.0001 mg to about 200 mg, or from about 0.001 mg to about 100 mg, or from about 0.01 mg to about 100 mg, or from about 0.1 mg to about 100 mg, or from about 1 mg to about 50 mg per kilogram of subject bodyweight.

### Kit of Parts

Compositions, their components, their formulation, and their dosage as described elsewhere in this specification are incorporated by reference herein and may be used to further describe the contents of the following kits.

A kit may have use at least some of its contents in methods as described here (e.g., methods for treatment and/or diagnosis). A kit may be comprised of a plurality of components, an optional dispenser of one or more components, a package, an optional package insert describing the components and their use, an optional seal that at least resists tampering with the components, an optional expiration date, and an optional wrapper. The package comprises any casing (e.g., carton, sack) that restricts access to its contents (e.g., one or more components) prior to opening. The casing may resist crushing. After the casing has been opened, it might be closed to restrict access to any remaining contents. It may or may not be possible to repeat the cycle of opening and closing the casing many times. The package may comprise an optional dispenser. An optional package insert may comprise information such as, for example, how to use a medicament, dosing and route of administration, risks and benefits of treatment, citation of medical literature and discussion of clinical studies, any drug interactions or other adverse effects, instructions for a companion diagnostic test, how to select subjects for treatment according to analysis of the test assay's result, or any combination thereof that has been approved by and/or complies with the requirements of a regulatory authority (e.g., U.S. FDA, European Medicines Agency). An optional seal may comprise a tamper-resistant adhesive label and/or a hologram attached to the package itself or some of the kit. When the package is opened and its contents are accessed, this would break the seal and the broken seal would be evidence of tampering. If the seal is intact, the user would be assured that no one has tampered with the components. An optional expiration date may comprise a date in the future printed on a label attached to the kit or the package itself, preferably not on any part of an optional wrapper that is discarded after unwrapping the package and before using its contents.

Kits may be manufactured by encasing at least the plurality of components, an optional dispenser, and an optional package insert. They may be inserted into a casing that is open or a casing may be assembled with them therewithin. During manufacture, at least one component (or at least one dispenser holding one or more components) may be secured in a compartment of the package. For kits capable of use over several days of treatment and/or a companion diagnostic test, multiple daily dosages of medicament and/or at least one diagnostic reagent may be secured in separate compartments with at least one component secured separate from another secured copy of the component. Alternatively, a dispenser having multiple daily dosages of medicament may be secured in its own compartment. In any order, the optional seal may be applied, the optional expiration date may be added, and the package may be wrapped. Preferably, the wrapper is transparent and the package (possibly, also the seal and/or the optional expiration date) is visible from outside. The optional seal may be applied to some part of the optional dispenser, the casing, the package, and/or the optional wrapping (which itself may comprise or consist of the seal). The package may be optionally wrapped for protection at least during storage, from the environment, during rough handling or transporting, or any combination thereof. The package and/or its wrapper may have printing at least identifying the kit, its source of origin (e.g., the manufacturer or its country of manufacture), the contents (e.g., a medicament or a diagnostic reagent), other information (e.g., URL or TCP/IP address of the manufacturer), or any combination thereof. When holding a wrapped package, the optional seal and/or the optional expiration date may be visible to an outside observer.

In some embodiments, kits are comprised of (i) a medicament comprising a THC compound (e.g., isolated or pure THC) and a melatonin compound (e.g., melatonin), optional polyphenolic compounds such as a curcumin-related compound (e.g., curcumin, turmeric) and a rutin-related compound (e.g., rutin), an optional Vitamin E-related compound, an optional at least one chemotherapeutic agent, pharmaceutically-acceptable excipients, an optional pharmaceutically-acceptable vehicle, and an optional pharmaceutically-acceptable carrier in solid, semisolid, or liquid form as one and only pharmaceutical composition (e.g., a single unit dose or a plurality of unit doses), in at least two different and distinct pharmaceutical compositions, in at least three different and distinct pharmaceutical compositions, in at least four different and distinct pharmaceutical compositions, or in at least five different and distinct pharmaceutical compositions, which may be administered in one or more doses of a daily dosage; (ii) an optional dispenser of one or more medicaments; (iii) a sealed package; (iv) a package insert comprising prescription information (e.g., how to use the medicament, dosing and route of administration, benefits and risks of treatment, citation of medical literature and discussion of clinical studies, any drug interactions or other adverse effects) that has been approved by and/or complies with the requirements of a regulatory authority (e.g., FDA, EMA); (v) a tamper-resistant seal; (vi) an expiration date of the medicament; and (vii) an optional wrapper. An optional dispenser may be any of, without limitation, a sealed blister pack (e.g., a blister card having capsules or tablets, a blister pouch having a cream or an ointment, a strip of detachable blister pouches having creams or ointments), a container having sealed ampules or sealed vials, a sealed container having capsules or tablets (e.g., pill bottle, pillbox), or the like. An optional dispenser (e.g., a capped bottle) containing multiple unit doses in liquid form may further comprise an optional tool (e.g., dropper, pipet) for measuring a volume of one or more unit doses. The sealed package may comprise the optional dispenser. Descriptions of the kit and its manufacture in the preceding two paragraphs are incorporated by reference herein.

In other embodiments, kits are comprised of (i) a plurality of compounds as described herein (e.g., a THC compound and a melatonin compound, optional polyphenolic compounds such as a curcumin-related compound and a rutin-related compound, an optional Vitamin E-related compound, an optional at least one chemotherapeutic agent, pharmaceutically-acceptable excipients, an optional pharmaceutically-acceptable vehicle, an optional pharmaceutically-acceptable carrier), which may be a medicament comprising such compounds formulated in solid, semisolid, or liquid form as a pharmaceutical composition (e.g., a single unit dose or a plurality of unit doses); (ii) an optional dispenser of one or more compounds or medicaments; (iii) at least one diagnostic reagent (e.g., a set of test assay reagents) and an optional container for collecting a sample (e.g., biofluid, tissue); (iv) a sealed package; (v) a package insert comprising prescription information for the medicament and an optional package insert for the test assay including use as a companion diagnostic device; (vi) a tamper-resistant seal; (vii) an expiration date of the medicament and an optional expiration date of the at least one diagnostic reagent; and (viii) an optional wrapper. A plurality of unit doses may be provided in an optional dispenser selected from, without limitation, a sealed blister pack (e.g., a blister card having capsules or tablets, a blister pouch having a cream or an ointment, a strip of detachable blister pouches having creams or ointments), a container having sealed ampules or sealed vials, a sealed container having capsules or tablets (e.g., pill bottle, pillbox), or the like. The sealed package may comprise the optional dispenser, a container having sealed ampules or sealed vials, a sealed container having capsules or tablets (e.g., pill bottle, pillbox), or the like. An optional dispenser (e.g., a capped bottle) containing multiple unit doses in liquid form may further comprise an optional tool (e.g., dropper, pipet) for measuring a volume of one or more unit doses. If a medicament and a diagnostic reagent are secured in compartments of the package, it is preferred that they be in separate compartments. Descriptions of the kit and its manufacture incorporated in the preceding paragraph are also incorporated by reference herein.

For kits comprising a medicament, reminders or a use instruction may be provided as indicia (e.g., letter, numeral, pictogram or logogram) printed on the medicament itself or adja-cent to an optional compartment securing the medicament. Reminders or the use instruction may correspond to a dosing schedule: e.g., the number and/or the order of doses administered within a time period (e.g., per day), the times of day or the days of the week when a dose should be administered, and the like. A dosage may be provided in kits for administration within a time period (e.g., daily) as a single unit dose, a plurality of unit doses all at a single time (e.g., first unit dose comprising a THC compound and second unit dose comprising a melatonin compound), or a plurality of unit doses at different times (e.g., first unit dose comprising a THC compound in the morning and second unit dose comprising a melatonin compound in the evening). All of the unit doses administered within 24 hours may constitute a daily dosage.

The optional at least one chemotherapeutic agent may be a drug used to treat dementia or Alzheimer's disease (e.g., aducanumab, amantadine, bepranemab, brexpiprazole, bryostatin, colostrinin, donanemab, donepezil, Ginko biloba extract EGb 761, galantamine, gantesnerumab, gosuranemab, lanabecestat, lecanemab, leucomethylthioninium, levetiracetam, memantine, methylphenidate, neflamapimod, prasinezumab, rivastigmine, semorinemab, solanezumab, tilavonemab, tramiprosate, tricaprilin, zagotenemab, or any combination thereof). The optional at least one chemotherapeutic agent may be selected from, without limitation, acetylcholinesterase inhibitors, antibodies binding beta-amyloid, antibodies binding tau protein, beta-amyloid aggregation inhibitors, beta-secretase inhibitors, gamma-secretase inhibitors, kinase inhibitors, receptor antagonists, tau phosphorylation inhibitors, tau protein aggregation inhibitors, or any combination thereof.

THC and melatonin compounds as described herein, optional two or more polyphenolic compounds (e.g., curcumin and rutin, compounds related thereto), optional Vitamin E-related compound, and optional at least one chemotherapeutic agent may be provided in two or more separate compartments of the kit. Alternatively, they may be provided together in the same compartment of the kit. Suitable packages and additional articles for use (e.g., premeasured vehicle and/or carrier for a semisolid or liquid form, foil wrapping to minimize exposure to air and moisture) are known in the art and the kit may further comprise them. Kits may comprise a blister pack containing multiple unit doses as a blister card or a strip of separable blister pouches. Kits may comprise a container having multiple unit doses, each in a sealed ampule or a sealed vial, a leak-resistant bag or bottle having liquid contents (e.g., colloid, elixir, hydrogel, tincture), a sealed container (e.g., pill bottle, pillbox) having capsules or tablets, or the like. Kits may further comprise a dispenser used to administer at least the THC and melatonin compounds as described herein, optional two or more polyphenolic compounds (e.g., curcumin and rutin, compounds related thereto), optional Vitamin E-related compound, and optional at least one chemotherapeutic agent. Examples of dispensers include, but are not limited to, dressings and patches, droppers and pipets, inhalers, insufflators, intravenous (IV) drips, misters and sprayers, nebulizers, suppositories, and vaporizers. The kits can be provided to healthcare providers (e.g., pharmacists, physicians, nurses) and caregivers.

A blister card for solid forms (e.g., capsules, tablets) may comprise a sheet of stiff but deformable material, preferably transparent, with depressions formed thereinto having the size and shape of the solid forms that are positioned in those depressions. The blister card may also comprise a sheet of tearable material, preferably coated paper or metal foil. Both sheets have substantially the same size and shape. They are aligned, pressed together, and sealed around the edge of each depression to form opposed top and bottom sides of the blister card with a single solid form being secured in each depression. As a result, the solid forms are sealed into the blister card. By manually pressing on any depression of the first sheet, that depression is flattened, a solid form is pushed until meeting resistance from the second sheet, that solid form tears an opening in the second sheet, and the solid form is removed through the opening without disturbing the solid forms in other depressions.

A blister pouch for a semisolid or liquid form (e.g., colloids, creams, hydrogels, suspensions) may comprise tearable material, preferably coated paper or metal foil, which may be one of multiple pouches arranged in a strip. For example, each pouch may be filled with a unit dose then sealed. As a result, a strip of blister pouches may contain a single unit dose of semisolid or liquid in each blister pouch. Packets in the strip may be separated from the next by tearing through a line of perforations. Each packet has a notch that can be torn to unseal the packet. The composition can be dispensed by tearing open a packet at its notch, flattening the packet, and expelling the semisolid or liquid from the opening.

Kits can further comprise a pharmaceutically-acceptable vehicle and/or a pharmaceutically-acceptable carrier that can be used to administer THC and melatonin compounds as described herein. For example, if the THC and melatonin compounds as described herein are provided in solid form, they must be reconstituted with a sterile vehicle and/or a sterile carrier prior to administration. The kit may further comprise a sealed container in which the THC and melatonin compounds as described herein are transported and where they can be optionally reconstituted with a sterile vehicle that is suitable for administration. Examples of pharmaceutically-acceptable vehicles include, but are not limited to, Water For Injection and other aqueous solutions (e.g., Sodium Chloride Injection, Ringer's Injection, Dextrose Injection, Dextrose and Sodium Chloride Injection, Lactated Ringer's Injection). Examples of sealed containers include, but are not limited to, ampules, bags, a blister card, blister pouches, bottles, a pillbox, and vials.

Inheritance of a genetic marker associated with increased risk of disease may be determined as step of a method for diagnosis or treatment. The genes of interest include, but are not limited to, APOE, APP, PSEN1, PSEN2, ABCA7, ADAM10, AKAP9, BIN1, CASS4, CD2AP, CD33, CELF1, CLU, CR1, CYP2C9, CYP3A4, DSG2, EPHA1, FERMT2, HLA-DRB5-HLA-DRB1, INPP5D, MAPT, MEF2C, MS4A6A/MS4A4E, NME8, PICALM, PLD3, PTK2B, SLC24A4, SORL1, TREM2, UNC5C, ZCWPW1, or any combination thereof. Strikingly, early-onset Alzheimer's disease may be caused by rare mutations in the foregoing genes or others newly discovered. A subject may be selected for treatment by the presence or absence of a gene mutation associated with increased or decreased risk for developing Alzheimer's disease.

Testing for a gene of interest such as, but not limited to, those listed in the previous paragraph may be done in a genetic testing laboratory, preferably AABB-accredited or CLIA-certified. Genetic testing may be selected from, without limitation, detecting mutations or polymorphisms, epigenomics, gene expression, genetic profiling or genotyping, pharmaco-genomics or precision medicine, and transcriptomics using a diagnostic reagent (e.g., CRISPR nucleases and their RNA guides, nucleic acid primers flanking the region of interest and probes to detect the region of interest, nucleic acid polymerases) and equipment (e.g., nucleic acid synthesizer, fluorometer or spectrophotometer, microarray scanner, NGS or DNA sequencing system, nucleic acid amplifier, nucleic acid extraction system). For example, a patient suspected of having late-onset disease may be tested for APOE alleles while a patient suspected of having early-onset disease may be tested for an APP, PSEN1 or PSEN2 mutation. Presence or absence of an APOE4 allele or a mutation in one or more genes of interest might be used to select for patients who are likely to benefit from a treatment. Expression of CYP2C9, CYP3A4, CYP2C19, CYP1A1, CYP1A2, or any combination thereof may be tested. The effective amount of THC may be increased or decreased based on expression of cytochrome P450 isoenzymes. Gene editing may be used to transfer a human mutation associated with increased risk for or protection against development of a neurodegenerative disease or a neurologic disorder into a model organism to study action of the mutation or effects of treatment. Besides detection of APOE alleles by genotyping, they may be distinguished as different protein isoforms.

Neuropathological testing of central nervous system (CNS) tissue may include, without limitation, histopathology (e.g., dyes, stains), immunoassay (e.g., analytes or antigens, antibodies), and immunohistochemistry. While biofluids and biopsies from living patients might sometimes be examined, many tissues are acquired postmortem for autopsy to confirm clinical diagnoses. The result may guide treatment of the patient providing the biopsy or treatment of patients in the future. Testing may be performed in a neuropathology laboratory, preferably AABB-accredited or CLIA-certified. Pathology testing may at least identify, at least localize within tissue, or at least quantitate any of the following, without limitation, diffuse or dense core plaques, granulovacuolar degeneration, Hirano bodies, insoluble amyloid fibrils, neuritic plaques, synaptic loss, tau neurofibrillary tangles, tau paired-helical filaments, or any combination thereof. A test assay kit may comprise aresealable, leak-resistant container having fixative (e.g., formaldehyde) to preserve a tissue sample for pathologic examination. Stains or dyes (e.g., Bodian Protargol, Congo red, Gallyas-Braak silver, Thioflavin) and antibodies specifically binding Abeta or tau may be used to visualize amyloid plaques and neurofibrillary tangles. Histologic processing, staining, and immunohistochemistry may be automated in at least some steps. In parallel, slides having abnormal structures may be dissected, nucleic acids prepared from the areas with abnormal structures and controls, and genetic testing performed on the nucleic acids. For a test assay kit, at least one or more copies of a diagnostic reagent may be selected from, without limitation, Bodian Protargol stain, Congo red dye, Gallyas-Braak silver stain, Thioflavin T dye, anti-Abeta antibody, anti-tau antibody, and a leak-resistant container containing preservative for collecting a tissue sample of a subject. The effect of treatment to modify disease and/or development of disease may be determined by changes in histological features of Alzheimer's disease.

A clinical laboratory may use tests and assays known in the art to at least detect (e.g., the presence or the absence) or at least measure (e.g., the amount, the concentration, the level) one or more biomarkers in a sample of a patient's biofluid (e.g., plasma, serum, whole blood, urine, cerebral spinal fluid or CSF). See Ruan et al. (Mol Med Rep, 2016 October, 14: 3184-3198) and McGrowder et al. (Brain Sci, 2021 February, 11: 215); which are incorporated by reference herein for listings of biomarkers and their use to monitor development of disease and, reciprocally, the treatment's ability to modify the disease process. The level of beta-amyloid peptide (Abeta42), phosphorylated tau protein (P-tau), total tau protein (T-tau), neurofilament light chain (NfL), ratio of Abeta42/P-tau, ratio of Abeta42/T-tau, or ratio of Abeta42/Abeta40 in blood, urine, or CSF may be assayed. A reduced level of at least one or more of, but not limited to, Abeta40/Abeta42 in plasma; Abeta40, Abeta42, P-tau, T-tau, Abeta42/P-tau, or Abeta42/T-tau in CSF may be used as a surrogate biomarker to determine if treatment at least slows, stops, or reverses development of disease. The proteins may be at least identified, isolated, quantitated, or separated with specific antibodies or optional competition with their cognate antigens, bead or plate immunoassays, high performance liquid chromatography (HPLC), mass spectrometry (MS), or any combination thereof (e.g., HPLC-MS, HPLC-MS/MS). Besides quantitation of a biomarker (e.g., Abeta40, Abeta42, APOE4) by immunoassay, HPLC-tandem MS can be used for quantitation. See, for example, West et al. (Mol Neurodegener, 2021 May, 16: 30); which is incorporated by reference herein. The effect of treatment to modify disease and/or development of disease may be determined by quantitating a biomarker (e.g., Abeta42/Abeta40), especially a change therein associated with an increase or a decrease in risk for developing Alzheimer's disease. For a test assay kit, at least one or more copies of a diagnostic reagent may be selected from, without limitation, an anti-Abeta antibody, an anti-tau antibody, an anti-NfL antibody, an anti-APOE antibody, or any combination thereof; a leak-resistant container for collecting a sample from blood (with or without anticoagulant), urine, or CSF; a known quantity of Abeta40, Abeta42, P-tau, T-tau, NfL, APOE isoforms, or any combination thereof.

Brain imaging (or scanning) is a radiologic test assay including, but not limited to, computed tomography, magnetic resonance imaging (MRI), positron emission tomography (PET), and single photon emission computed tomography (SPECT). Brain atrophy, cerebral amyloid angiopathy, ventricular enlargement, and diffusion tensor imaging of white matter can be seen with structural MRI to monitor neurodegeneration. Synaptic activity (lower in disease) in the brain may be visualized using PET and fluorodeoxyglucose (FDG) reacting to glucose metabolism (hypometabolism). More interestingly, brain circuits and network activity may be assessed with functional MRI (fMRI) measuring changes in blood oxygenation level dependent (BOLD) contrast at rest. Probes for amyloid (e.g., florbetaben F-18, florbetapir F-18, flutemetamol F-18) and tau (e.g., flortaucipir F-18) may be used with PET to detect amyloid and tau pathology (e.g., cortical amyloid or tau PET). A change in brain imaging may be used to select patients for treatment (e.g., absence or presence of a change indicative of or associated with increased risk for Alzheimer's disease) or disease modification due to treatment using at least one or more copies of a diagnostic reagent such as, for example, FDG, PET and SPECT probes for amyloid and/or tau.

A test assay kit may comprise one or more diagnostic reagents (e.g., a set of test assay reagents) for use in one or more test assaying steps of a diagnostic method, including an optional enzyme, an optional plurality of standards for the test assay, an optional at least one or two controls (e.g., positive and/or negative controls that produce or do not produce, respectively, a signal in the test assay), or any combination thereof. The standards may contain known amounts of analyte and/or label to at least calibrate, confirm, or verify the test assay's ability to quantitate analyte. A test assaying step of a diagnostic method may be performed outside of a method of treatment by someone who is not administering a pharmaceutical composition or medicament to a subject (i.e., the claimed methods of treatment and administering the claimed products) and whose contact with a subject is, at most, obtaining a sample therefrom or the subject providing same. The result of the test assaying step or optional information associated with a sample (or a subject from whom the sample was obtained) may be used in an analyzing and/or a determining step to diagnose the subject, to select the subject for treatment, to assess or evaluate a treatment outcome (e.g., reducing, inhibiting worsening, or reversing a biomarker), or any combination thereof.

### Formulations for Enteral Administration

Compositions for enteral administration may comprise THC and melatonin compounds as described herein, optional polyphenolic compounds, and one or more pharmaceutically-acceptable excipients suitable for enteral administration. In some embodiments, compositions for enteral administration may comprise therapeutically effective amounts of THC and melatonin compounds as described herein; optionally, effective amounts of two or more polyphenolic compounds; one or more pharmaceutically-acceptable excipients; and optional pharmaceutically-acceptable vehicle and/or carrier suitable for enteral administration. Examples of suitable excipients include, but are not limited to, antimicrobials, antioxidants, emulsifiers, solubilizers, stabilizers, and surfactants. In other embodiments, compositions for enteral administration may further comprise an effective amount of at least one chemotherapeutic agent.

Compositions for enteral administration contain amounts of two or more compounds as described herein (e.g., THC compounds; melatonin compounds; optional polyphenolic compounds such as curcumin and rutin, optional Vitamin E, and compounds related thereto; optional at least one chemotherapeutic agent). They may be provided in the form of discrete, unit doses such as, but not limited to, capsules, chartulae or sachets, chewable candies or gums, disintegrating strips or tabs, lozenges or pastilles, or tablets. Multiple unit doses can be held in a dispenser such as, but not limited to, a sealed blister card containing medicaments (e.g., capsules, tablets), a sealed container containing medicaments (e.g., chewable candies or gums, disintegrating strips or tabs, lozenges or pastilles), and a sealed pill bottle or pillbox containing medicaments (e.g., capsules, tablets). Other enteral compositions may be provided in solid form (e.g., powders), semisolid form (e.g., paste, slurry) or liquid form such as, but not limited to, aerosols, colloids, drops or other ingestible solutions, elixirs, mouthwashes or oral rinses, suspensions, and tinctures. For enteral administration, the route of administration is usually peroral or esophageal, but other routes are possible if the composition is digested and at least some compounds are adsorbed in the gastrointestinal tract.

Compositions may be formulated by conventional processes as known in the art. For example, mixing uniformly and intimately together THC and melatonin compounds as described herein, optional two or more polyphenolic compounds, optional Vitamin E-related compound, optional at least one chemotherapeutic agent, and one or more pharmaceutically-acceptable excipients in an intermediate state of matter that may be in solid form (e.g., granules), semisolid form (e.g., paste, slurry), or liquid form. Compositions may further comprise a vehicle and/or a carrier. Formulation may further comprise mixing with at least a vehicle and/or a carrier, dispersing in a colloid or a suspension, encapsulating in particles, incorporating in micelles or lipid vesicles, solubilizing in an emulsion or a solution, or any combination thereof.

If necessary, filing a capsule with the compounds as described here or compressing a tablet out of them will formulate a unit dose from the unfinished composition. For example, capsules or tablets may be manufactured in an encapsulating or tableting machine, respectively, by providing free-flowing, granules or a fine powder (e.g., about 40-200 mesh) comprising a mixture of THC and melatonin compounds as described here, optional polyphenolic compounds, optional Vitamin E-related compound, optional at least one chemotherapeutic agent, and one or more pharmaceutically-acceptable excipients (e.g., preservatives, disintegrants, diluents or fillers) to fill empty capsules or to mold into tablets. Alternatively, providing a flowable solution, oil, or syrup will enable filling empty capsules (e.g., gelatin or hydroxypropyl methylcellulose shells) with a semisolid or liquid form (e.g., alcoholic water, glycerol, honey, oil) of a composition comprising THC and melatonin compounds as described here, optional polyphenolic compounds, optional Vitamin E-related compound, optional at least one chemotherapeutic agent, and one or more pharmaceutically-acceptable vehicles, carriers, and/or excipients (e.g., preservatives, emulsifiers, diluents or fillers). A capsule size of 000 (1.4 ml), 00 (0.9 ml), or 0 (0.7 ml) may be preferred. Optional excipients may be added to the capsule or tablet to chemically stabilize at least the THC and melatonin compounds as described herein during formulation and/or to physically stabilize the “finished” composition prior to its transportation and use in treatment. For example, at least the THC and melatonin compounds as described herein may be uniformly and intimately mixed with a finely divided solid carrier.

Capsules may be manufactured in solid or semisolid form by filling their empty shells with a mixture comprising THC and melatonin compounds as described herein, optional polyphenolic compounds (e.g., curcumin and rutin, compounds related thereto), optional Vitamin E-related compound, optional at least one chemotherapeutic agent, two or more pharmaceutically-acceptable excipients, and optional pharmaceutically-acceptable carrier in a machine for encapsulating. Glidants and lubricants may be used to aid the manufacturing process. If desired for improved storage or drug delivery, the capsule may be given one or more coatings to provide controlled, delayed, or sustained release. Glidants and lubricants may be used to aid the manufacturing process. An enteral composition in liquid form may be encapsulated as soft gelatin capsules in semisolid form. The capsule may comprise a hard or a soft shell, a shell made from gelatin or hydroxypropyl methylcellulose, an enteric coating, or any combination thereof.

Tablets may be manufactured in solid form by compacting or molding a composition comprising THC and melatonin compounds as described herein, optional polyphenolic compounds (e.g., curcumin and rutin, compounds related thereto), optional Vitamin E-related compound, optional at least one chemotherapeutic agent, two or more pharmaceutically-acceptable excipients, and optional pharmaceutically-acceptable carrier in a machine for tableting. Glidants and lubricants may be used to aid the manufacturing process. Disintegrants (e.g., crospovidone, hydroxypropyl methylcellulose) and solubilizers (e.g., cyclodextrins, polypropylene glycols) may be added to enhance drug delivery. If desired for improved storage or drug delivery, a multilayered tablet and/or a multicoated tablet can be manufactured to provide controlled, delayed, or sustained release. A matrix tablet or an enteric-coated tablet may target drug delivery.

Compounds as described herein can be combined with one or more pharmaceutically-acceptable vehicles, carriers, and/or excipients, then formulated using conventional processes as known in the art. The carrier can take a wide variety of forms depending on the form of composition desired for administration. When formulating enteral compositions for use perorally, any of the usual pharmaceutically-acceptable media can be employed as vehicles and/or carriers, such as, for example, alcohols, emulsions, glycerol, lipid vesicles, micelles, oils, particulates, or water in the case of enterally administered compositions in liquid form (e.g., colloids, elixirs, ingestible solutions, suspensions, tinctures). Excipients such as cellulose derivatives, microcrystalline or powdered cellulose, pregelatinized starches, starches, sugars, syrups, binders, diluents or fillers, and disintegrants can be used in the case of enterally administered, compositions in solid form. For example, suitable solid forms for enteral administration include, but are not limited to, capsules and tablets. In some embodiments, the solid form can be coated or uncoated, encapsulated in gelatin or another polymer, or distributed nonhomogeneously (e.g., multiple layers, each having different release characteristics, or embedded in the matrix of a tablet) by conventional processes as known in the art. The capsule or tablet is preferably enterically coated.

When a colloid, an elixir, an ingestible solution, a suspension, a tincture, or the like is desired for enteral administration, THC and melatonin compounds as described therein can be mixed with optional polyphenolic compounds (e.g., curcumin and rutin, compounds related thereto), optional Vitamin E-related compound, optional at least one chemotherapeutic agent, optional vehicle (e.g., ethanol, glycerol, water), optional carrier (e.g., lipid vesicles, micelles, nanoemulsions, nanoparticles), and excipients (e.g., preservatives, emulsifiers). An enteral composition in semisolid (e.g., paste, slurry) or liquid form may be encapsulated in soft gelatin capsules.

A composition may be formulated for enteral administration by combining a THC compound, a melatonin compound, a curcumin-related compound, a rutin-related compound, a Vitamin E-related compound, optional pharmaceutically-acceptable vehicle (e.g., alcoholic waters, alcohols, glycerol, vegetable oils), optional pharmaceutically-acceptable carrier (e.g., a nanoemulsion), and pharmaceutically-acceptable excipients including but not limited to, a preservative (e.g., ascorbic acid, glycerol, sodium benzoate), an emulsifier (e.g., glycerol, polysorbate 80, propylene glycol), a diluent or filler (e.g., calcium carbonate), a colorant (e.g., brown E150, titanium oxide, yellow E110), a flavorant (e.g., honey, menthol, sorbitol), and a buffer (e.g., sodium citrate). Capsules and tablets can be coated by conventional processes as known in the art, avoiding degradation in stomach acid and delaying release until past the stomach, to at least enhance bioavailability and stabilize the THC and melatonin compounds as described herein. Capsules may comprise shells of cellulose (e.g., hydroxypropyl methylcellulose), gelatin, shellac, or the like. Alternatively, capsules and tablets may comprise a coating of anionic polymethacrylates (e.g., Eudragit), cellulose acetate phthalate (e.g., Aquateric), polyvinyl acetate phthalate (e.g., Coaterir), glyceryl monostearate, glyceryl distearate, or the like. The coating on capsules or tablets may be the chief determinant of their release characteristics and bioavailability; preferably, the capsules or tablets comprise an enteric coating for delivery of THC and melatonin compounds as described herein into the gastrointestinal tract where they are absorbed. They are metabolized by hepatic enzymes before entering the systemic circulation.

Preservatives may be one or more of the following: antimicrobials, antioxidants, and stabilizers. Suitable antimicrobials include, but are not limited to, benzalkonium salts, benzoic acid, benzyl alcohol, boric acid, chloroxylenol, ethanol, glycerol, sodium benzoate, and sorbic acid. Suitable antioxidants include, but are not limited to, ascorbic acid, ascorbyl palmitate, beta-carotene, monothioglycerol, potassium bisulfite, potassium metabisulfite, sodium ascor-bate, sodium bisulfite, sodium metabisulfite, sodium sulfite, and tocopherols. Stabilizers include, but are not limited to, celluloses, citric acid, cyclodextrins, edetate disodium, ethylene-diaminetetraacetic acid, polyethylene glycols, polyols, propylene glycol, starches, and trehalose.

An emulsifier can be used to form compositions. Suitable emulsifiers are selected from, without limitation, hydrophilic surfactants and lipophilic surfactants. A mixture of hydrophilic surfactants can be employed, a mixture of lipophilic surfactants can be employed, or a mixture of at least one hydrophilic surfactant and at least one lipophilic surfactant can be employed. Hydrophilic surfactants may be ionic or nonionic.

Suitable ionic surfactants include, but are not limited to, acylacrylates; alkylammonium salts; alkylsulfate salts; carnitine fatty acid ester salts; citric acid esters of mono- and di-glycerides; fatty acid salts; fatty acid derivatives of amino acids, oligopeptides, and polypeptides; fusidic acid salts; glyceride derivatives of amino acids, oligopeptides, and polypeptides; lactylic esters of fatty acids; lysophospholipids and derivatives thereof; mono- and di-acetylated tartaric acid esters of mono- and di-glycerides; phospholipids and derivatives thereof; and succinylated mono- and di-glycerides. In particular, the ionic surfactant may be cholyl-sarcosine; lauroyl carnitine, palmitoyl carnitine, myristoyl carnitine, or a salt thereof; caprate, caproate, caprylate, laurate, linoleate, myristate, oleate, palmitate, or a salt thereof; PEG- or PVP-phosphatidylethanolamine; phosphatidic acid, phosphatidylcholine, phosphatidylethanolamine, phosphatidylglycerol, or phosphatidylserine; lysophosphatidylcholine, lysophosphatidylethanolamine, lysophosphatidylglycerol, lysophosphatidic acid, or lysophosphatidylserine; sodium docusate; and stearoyl-2-lactylate or stearoyl lactylate.

Hydrophilic, nonionic surfactants include, but are not limited to, polyethylene glycol alkyl ethers, polyethylene glycol alkyl phenols, polyethylene glycol fatty acid monoesters, polyethylene glycol fatty acid diesters, polyethylene glycol glycerol fatty acid esters, polyglycerol fatty acid esters (e.g., polyglyceryl-3 oleate, polyglyceryl-10 laurate, poly-glyceryl-10 oleate), polyethylene glycol (e.g., PEG 200, PEG 400, PEG 600, PEG 1000), polyoxyethylated vitamins (e.g., alpha-tocopheryl PEG 1000 succinate), polyoxyethylene sterols, polyoxyethylene-polyoxypropylene block copolymers (e.g., poloxamer 188, poloxamer 238), polyethylene glycol sorbitan fatty acid esters, polysorbates (e.g., polysorbate 20, polysorbate 40, polysorbate 60, polysorbate 80), sorbitan fatty acid esters (e.g., sorbitan monolaurate, sorbitan monooleate), sucrose monostearate, sucrose monolaurate, and sucrose monopalmitate.

Suitable lipophilic surfactants include, but are not limited to, fatty alcohols, glycerol fatty acid esters, acetylated glycerol fatty acid esters, propylene glycol fatty acid esters, sorbitan fatty acid esters, polyethylene glycol sorbitan fatty acid esters, sterols and their derivatives, polyoxyethylated sterols and their derivatives, polyethylene glycol alkyl ethers, and oil-soluble vitamins or their derivatives. Other examples of lipophilic surfactants include, but not limited to, glycerol fatty acid esters, propylene glycol fatty acid esters, and hydrophobic transesterification products of a polyol with a vegetable oil or triglyceride (e.g., Cremophor or Kolliphor).

A binder may be used to formulate compositions in solid form by binding together disparate components. Examples of suitable excipients that may be used as binders include, but are not limited to, alginates, alginic acid, carrageenan, cellulose and derivatives thereof (e.g., carboxymethyl cellulose, cellulose acetate, ethylcellulose, hydroxyethyl cellulose, hydroxypropyl cellulose hydroxypropyl methylcellulose, powdered cellulose, microcrystalline cellulose), crosslinked polyvinyl pyrrolidones, gelatin, guar gum, gum tragacanth, polyvinyl pyrrolidones, pregelatinized starches, starches, and xanthum gum.

A diluent or filler may be used to formulate compositions in solid form to achieve a suitable size. This excipient may be considered a bulking agent because it bulks up a small amount of therapeutically active compound during formulation and is a major contributor to the volume of the finished unit dose. Examples of suitable excipients that may be used as diluents or fillers include, but are not limited to, aluminum silicate, calcium carbonate, calcium phosphate dibasic dihydrate, dextrates, lactose, magnesium silicate, magnesium stearate, mannitol, microcrystalline cellulose, powdered cellulose, pregelatinized starches, sorbitol, sucrose, and starches.

A disintegrant may be used to facilitate disintegration of solid forms (e.g., capsule, tablet) of a composition when exposed to the gastrointestinal tract. Too much disintegrant can produce a solid form that is easily broken during transport, while too little can be insufficient for disintegration to occur. Therefore, using an amount of disintegrant outside the operating range may alter the rate and extent of release of one or more of the compounds as described herein. Assuming enteral administration is peroral, the amount of disintegrant is sufficient to enable manufacturing a solid form of the composition and to be substantially digestible, which may depend on the chemical properties of other excipients and any coating. Examples of suitable excipients that may be used as disintegrants include, but are not limited to, alginic acid, calcium carbonate, calcium phosphate dibasic dihydrate, carboxymethyl cellulose, crosslinked polyvinyl pyrrolidones, guar gum, hydroxypropyl methylcellulose, methylcellulose, microcrystalline cellulose, polacrilin potassium, polyvinyl pyrrolidones, sodium starch glyco-late, and starch.

Glidants and lubricants are used during the manufacture of solid forms (e.g., capsules, tablets) of a composition to facilitate the flow of anhydrous or low-moisture ingredients within a machine or to prevent capsules and tablets from sticking to the machine's surfaces, respectively. Glidants smooth and even out the filling of empty shells (then having their contents sealed to form capsules) and die cavities (then having their contents compacted to form tablets) by reducing friction between particulates. Suitable glidants may be selected from, but are not limited to, ascorbyl palmitate, ascorbyl stearate, calcium palmitate, colloidal silica, magnesium carbonate, magnesium silicate, magnesium stearate, starch, and talc. Lubricants ease removing newly manufactured capsules and tablets from where they are held in the machine by reducing friction between them and the machine's surfaces. Suitable lubricants may be selected from, but are not limited to, calcium stearate, glyceryl behenate, magnesium stearate, polyethylene glycol, sodium lauryl sulfate, stearic acid, and zinc stearate. Glidants and lubricants, along with antiadherent excipients, may be considered lubricating agents. Generally, glidants and lubricants will only be used in small amounts during the manufacture of solid forms of the composition, and they might not carry over to the final product.

Compositions can include a solubilizer to ensure adequate dispersion or dissolution of two or more compounds as described herein (e.g., THC compounds; melatonin compounds; polyphenolic compounds such as curcumin and rutin, Vitamin E, and compounds related thereto; at least one chemotherapeutic agent) and to minimize precipitation of any compounds. This can be especially important for compositions for nonoral use (e.g., compositions for injection). A solubilizer can also be added to increase the solubility of hydrophilic compounds and/or other components, such as surfactants, or to maintain the composition as a stable or homogeneous solution.

Suitable solubilizers may be selected from alcohols and polyols (e.g., benzyl alcohol, cyclodextrins, diethylene glycol monoethyl ether, polyethylene glycols, polypropylene glycols, polyvinyl alcohols, propylene glycol), amides and other nitrogen-containing compounds (e.g., N-alkyl pyrrolidones, N-hydroxyalkyl pyrrolidones, 2-pyrrolidone, 2-piperidone, polyvinyl pyrrolidones), and esters (e.g., acetyl tributyl citrate, acetyl triethyl citrate, beta-butyrolactone, delta-valerolactone, ethyl butyrate, ethyl caprylate, ethyl oleate, ethyl propionate, glyceryl triacetate, N-2-hydroxyethyl-2-pyrrolidone, N-methyl-2-pyrrolidone, propylene glycol diace-tate, propylene glycol monoacetate, tributylcitrate, triethyl citrate). Further examples of suitable solubilizers include, but are not limited to, benzyl alcohol, diethylene glycol monoethyl ether, glycerol, glyceryl triacetate, PEG 400, polysorbate 80, and propylene glycol. A suitable amount of solubilizer can be readily ascertained by a person skilled in the art. In some circumstances, it can be advantageous to include amounts of solubilizers far in excess of pharmaceutically-acceptable amounts, for example to maximize the concentration of the drug, with excess solubilizer removed prior to providing the composition to a subject using conventional processes (e.g., distillation, evaporation) as known in the art.

Solvents for hydrophobic compounds include, but are not limited to, alcohols, fatty acid esters, fatty alcohols, glycerol, mineral oil, silicone oils, and vegetable oils. Vegetable oils are made from plants such as, without limitation, avocado, babassu, cacao, candlenut, castor, coconut, corn, hazelnut, macadamia, olive, palm, palm kernel, peanut, rapeseed, rice bran, safflower, sesame, soybean, sunflower, sweet almond, walnut, and wheat germ. Carnauba wax, cocoa butter, jojoba wax, mango butter, paraffin wax, shea butter, silicone wax, soy wax, stearyl alcohol, and white petrolatum may be used to formulate ointments, suspensions, and the like.

In addition, a base and/or an acid can be incorporated into the composition to facilitate processing, to enhance stability, or for other reasons. Examples of pharmaceutically-acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris-(hydroxymethyl)-aminomethane, and the like. Other suitable bases are salts of a pharmaceutically-acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid. Salts of polyprotic acids, such as sodium phosphate, disodium hydrogen phosphate, and sodium dihydrogen phosphate can also be used. When the base is a salt, its cation can be any pharmaceutically-acceptable cation, such as ammonium, an alkali metal (e.g., lithium, potassium, sodium), or an alkaline earth metal (e.g., calcium, magnesium).

Acids may be organic or inorganic acids. Examples of pharmaceutically-acceptable inorganic acids include hydrochloric acid, hydrobromic acid, hydriodic acid, sulfuric acid, nitric acid, boric acid, phosphoric acid, and the like. Examples of pharmaceutically-acceptable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like.

### Formulations for Parenteral Administration

Compositions for parenteral administration may comprise THC and melatonin compounds as described herein, optional polyphenolic compounds, optional Vitamin E-related compound, and one or more pharmaceutically-acceptable excipients suitable for parenteral administration. In some embodiments, compositions for parenteral administration may comprise therapeutically effective amounts of THC and melatonin compounds as described herein; optionally, effective amounts of two or more polyphenolic compounds; optionally, effective amount of Vitamin E-related compound; and one or more pharmaceutically-acceptable vehicles, carriers, and/or excipients suitable for parenteral administration. Examples of excipients include, but are not limited to, preservatives (e.g., antimicrobials, antioxidants, stabilizers); emulsifiers, solubilizers, and surfactants. In other embodiments, compositions for parenteral administration may further comprise an effective amount of at least one chemotherapeutic agent.

A parenteral composition in liquid form (e.g., an infusible or injectable solution) may be administered by infusion or injection. If a parenteral composition is in solid form, it may be reconstituted under aseptic conditions with a pharmaceutically-acceptable vehicle, preferably sterile, as an aerosol, a colloid, a cream, a foam or a mousse, a hydrogel, a solution, or a suspension. Excipients such as emulsifiers, solubilizers, surfactants, antifoaming agents, and wetting agents may be included during formulation of the parenteral composition.

Compositions may comprise at least one surfactant selected from polysorbates, sorbitan esters, polyoxyl stearate, and polyoxyethylene-polyoxypropylene block copolymers. Surfactants also include polyoxyethylene esters of sorbitan fatty acids (e.g., PEG sorbitan monooleate) and polyoxyethylated vitamins (e.g., alpha-tocopheryl PEG 1000 succinate), sorbitan fatty acid esters (e.g., sorbitan monooleate), or any combination thereof. Other suitable excipients (e.g., preservatives, antimicrobials, antioxidants, stabilizers, emulsifiers, solubilizers, surfactants, diluents or fillers) are as described above for enteral compositions. Those excipients and their descriptions are incorporated by reference herein.

Pharmaceutically-acceptable vehicles for parenteral administration may be sterile aqueous solutions such as, for example, Water For Injection USP, Sodium Chloride Injection, Ringer's Injection, Dextrose Injection, Dextrose and Sodium Chloride Injection, and Lactated Ringer's Injection but other physiological saline or buffered, isotonic solutions might also be suitable. The parenteral composition may include excipients such as, but not limited to, one or more of albumin, gelatin, cellulose and derivatives thereof (e.g., carboxymethyl cellulose, ethyl cellulose, hydroxyethyl cellulose, hydroxypropyl cellulose, hydroxypropyl methylcellulose, methylcellulose, microcrystalline cellulose, powdered cellulose), buffering agents (e.g., sodium acetate, sodium citrate, sodium phosphate); tonicity agents (e.g., dextrose, potassium salts, sodium salts), polyols (e.g., crosslinked polyacrylates, glycerol, polyethylene glycols, polypropylene glycols, propylene glycol, sodium polyacrylates), polysaccharides (e.g., chitosan, chondroitin sulfate, hyaluronic acid, lactose, maltose, mannitol, sodium hyaluronate, sorbitol, trehalose), polyvinyls (e.g., carboxyvinyl polymers, crosslinked polyvinyl pyrrolidone, polyvinyl alcohols, polyvinyl pyrrolidone), preservatives (e.g., benzalkonium chloride, benzethonium chloride, benzyl alcohol, chloroxylenol, ethanol, sodium benzoate), emulsifiers (e.g., glycerol, polysorbate 80, propylene glycol), or any combination thereof.

Parenteral compositions may be provided as a single unit dose in an ampule, a blister pouch, a dropper bottle, a vial, or the like, each containing amounts of two, three, four, five, six, or more compounds (e.g., THC compounds; melatonin compounds; polyphenolic compounds such as curcumin and rutin, Vitamin E, and compounds related thereto; at least one chemotherapeutic agent) as described herein. Alternatively, parenteral compositions comprising two or more compounds as described herein (e.g., THC compounds; melatonin compounds; optional polyphenolic compounds such as curcumin and rutin, optional Vitamin E, and compounds related thereto; optional at least one chemotherapeutic agent) can be formulated into a semisolid or liquid form such as, but not limited to, aerosols, colloids, creams, foams or mousses, hydrogels, solutions, and suspensions. Parenteral composition may be dispersed in a colloid or a suspension, encapsulated in particles, incorporated in micelles or lipid vesicles, or solubilized in an emulsion or a solution. Formulation may further comprise mixing with at least a vehicle and/or a carrier, dispersing in a colloid or a suspension, encapsulating in particles, incorporating in micelles or lipid vesicles, solubilizing in an emulsion or a solution, or any combination thereof.

The route of parenteral administration may be mucosal (e.g., buccal, nasopharyngeal, sublabial, sublingual), pulmonary (e.g., inhalation, insufflation, instillation), or any combination thereof. Multiple unit doses can be held in a dispenser such as, but not limited to, sealed ampules or sealed vials containing medicament (e.g., injectable solution), a sealed bag or bottle containing medicament (e.g., colloid, drops, infusible solution, suspension), and a sealed tub or tube containing medicament. Parenteral administration may be by means of a dispenser such as, but not limited to, an inhaler, an insufflator, a mister or a sprayer, a nebulizer, and a vaporizer.

### Formulations for Pulmonary Administration

Compositions for pulmonary administration may comprise THC and melatonin compounds as described herein, optional polyphenolic compounds, optional Vitamin E-related compound, optional at least one chemotherapeutic agent, and one or more pharmaceutically-acceptable excipients suitable for pulmonary administration. In some embodiments, pulmonary compositions may comprise therapeutically effective amounts of THC and melatonin compounds as described herein; optionally, effective amounts of two or more optional polyphenolic compounds such as curcumin and rutin, Vitamin E, and compounds related thereto; optional effective amount of at least one chemotherapeutic agent; and one or more pharmaceutically-acceptable vehicles, carriers, and/or excipients suitable for pulmonary administration. Examples of excipients include, but are not limited to, preservatives, antimicrobials, antioxidants, and stabilizers; emulsifiers, solubilizers, and surfactants. Other suitable excipients are as described above for enteral and parenteral compositions; those excipients and their descriptions are incorporated by reference herein.

Pulmonary compositions may be in solid form (e.g., granules) or liquid form. If in solid form, they may or may not be reconstituted as liquids prior to administration. In some embodiments, they may be aerosols, colloids, drops, or suspensions in which THC and melatonin compounds as described herein are formulated (e.g., dissolved, dispersed, emulsified, encapsulated, suspended) in a pharmaceutically-acceptable vehicle (e.g., aqueous solution, organic solvent). A composition in liquid form or as a solution reconstituted with a sterile vehicle from solid form may further comprise a pharmaceutically-acceptable carrier selected from an emulsion (e.g., a nanoemulsion), micelles or lipid vesicles, porous or solid particles (e.g., nanoparticles), or any combination thereof. Pulmonary compositions may be administered by inhalation or insufflation via an oral or nasal route for a systemic effect.

Pulmonary administration of the composition may be inhalation or insufflation through the mouth and/or nose. In particular, a dispensing device may be used to produce aerosols from an inhaler, a mister or a sprayer, a nebulizer, or a vaporizer. The “finished” composition may be provided in solid form (e.g., granules) or a liquid form, which is aerosolized during pulmonary administration such that the aerosol particles or droplets, respectively, are administered into the mouth and/or nose. The goal of pulmonary administration is to deliver the composition from the upper respiratory tract into the lower respiratory tract where THC and melatonin compounds as described herein; optional polyphenolic compounds; optional Vitamin E-related compound, two or more pharmaceutically-acceptable excipients; optional at least one chemotherapeutic agent; and optional vehicle and/or carrier are absorbed by the alveolar epithelium because of the lung's unique physiological advantages. Alternatively, a liquid composition can be more efficiently delivered by instilling the composition directly into the trachea. Pulmonary administration may provide a local effect in the lungs or a systemic effect by adsorption though respiratory mucosa directly into the circulation, bypassing first pass hepatic metabolism.

### Formulations for Controlled Time Release Administration

Compositions for controlled time release administration (e.g., delayed, extended, sustained) may comprise THC and melatonin compounds as described herein, optional polyphenolic compounds, optional Vitamin E-related compound, and one or more pharmaceutically-acceptable excipients suitable for controlled time release administration. In some embodiments, compositions for controlled time release may comprise therapeutically effective amounts of THC and melatonin compounds as described herein; optionally, effective amounts of two or more polyphenolic compounds; optionally, effective amount of Vitamin E-related compound; and one or more pharmaceutically-acceptable vehicles, carriers, or excipients suitable for controlled time release administration. Examples of excipients include, but are not limited to, preservatives, antimicrobials, antioxidants, and stabilizers; emulsifiers, solubilizers, and surfactants. In other embodiments, compositions for controlled time release administration may further comprise an effective amount of at least one chemotherapeutic agent, which may be a drug used to treat dementia or Alzheimer's disease (e.g., aducanumab, amantadine, bepranemab, brexpiprazole, bryostatin, colostrinin, donanemab, donepezil, Ginko biloba extract EGb 761, galantamine, gantesnerumab, gosuranemab, lanabecestat, lecanemab, leucomethylthioninium, levetiracetam, memantine, methylphenidate, neflamapimod, prasinezumab, rivastigmine, semorinemab, solanezumab, tilavonemab, tramiprosate, tricaprilin, zagotenemab, or any combination thereof). They may be selected from, without limitation, acetylcholinesterase inhibitors, antibodies binding beta-amyloid, antibodies binding tau protein, beta-amyloid aggregation inhibitors, beta-secretase inhibitors, gamma-secretase inhibitors, kinase inhibitors, receptor antagonists, tau phosphorylation inhibitors, and tau protein aggregation inhibitors. Systemic effects may be achievable thereby.

Controlled time release compositions may be administered by delayed, extended, sustained, or other controlled time release using a carrier such as, but not limited to, an emulsion, micelles or lipid vesicles, a nanoemulsion, nanoparticles, solid or porous particles, or other conventional means for controlled time release (e.g., enteric coating of the composition in solid form) as known in the art. Absorption of an infused or injected composition can be controlled by including an excipient that delays absorption. Controlled time release may use a dispenser such as, for example, an infusion pump. Suitable controlled time release formulations are known in the art, including those described herein, to provide a desired release profile of the compounds by using unit doses suitable for enteral administration such as, but not limited to, capsules and tablets that are adapted for controlled time release by coatings or multilayer construction.

Controlled time release compositions may improve treatment over that achieved by noncontrolled time release compositions. In some embodiments, the use of a controlled time release formulation in treatment is characterized by a minimum of the THC and melatonin compounds as described herein being administered to treat the neurodegenerative disease or neurologic disorder in a minimum amount of time. Advantages of controlled time release compositions include at least increased bioactivity or bioavailability of the compounds, reduced dosage frequency, or improved patient compliance with the treatment protocol. In addition, controlled time release compositions can be used to affect the time of onset of action or other characteristics, such as blood levels of the drug, and can thus affect the occurrence of adverse side effects.

Controlled time release compositions may be designed to initially release effective amounts of the compounds as described herein (e.g., THC compounds; melatonin compounds; optional polyphenolic compounds such as curcumin and rutin, optional Vitamin E, and compounds related thereto; optional at least one chemotherapeutic agent) that promptly produces at least the desired therapeutic, prophylactic, preventive, palliative, or other beneficial effect in a loading phase, and gradually releases lower amounts of the compounds as described herein to maintain this level of beneficial effect over an extended period in a maintenance phase. To maintain effective levels of THC and melatonin compounds as described herein, optional polyphenolic compounds, optional Vitamin E-related compounds, and optional at least one chemotherapeutic agent in the body, they can be released at a rate that will replace the amounts that are being metabolized and excreted from the body. Controlled time release of compounds as described herein can be changed by various conditions including, but not limited to, pH, salts, temperature, and other physiological conditions. For example, an enteric capsule or tablet has a polymeric coating that degrades in response to pH in the gastrointestinal tract. Alternatively, instead of an enteric-coated capsule, the capsule shell may itself be made of enteric material or the capsule contents may be particulate, having enteric coating on the particles.

Compositions can be administered using an infusion pump, a transdermal patch having penetration enhancers, a nanoemulsion, polymeric or metallic nanoparticles, micelles or lipid vesicles, or other means for controlled release administration. See Pons-Faudoa et al. Biomed Microdevices, 2019 May, 21: 47; Rahnfeid & Luciani Pharmaceutics, 2020 June, 12: 0567; Nkanga et al. Adv Drug Deliv Rev, 2020 December, 167: 19-46. In another embodiment, implanted polymeric materials or injectable depots may be placed at an appropriate site in a subject to deliver the composition. THC and melatonin compounds as described herein may diffuse from a solid matrix and enter the systemic circulation in a controlled releasing step.

Reduced degradation of the compounds as described herein (e.g., THC compounds; melatonin compounds; optional polyphenolic compounds such as curcumin and rutin, optional Vitamin E, and compounds related thereto; optional at least one chemotherapeutic agent) and/or their increased absorption enterally may also be achieved by encapsulating them in lipid vesicles. In a distribution of sizes within the population, 90% or more of the lipid vesicles may or may not have a diameter of less than 500 nm (e.g., more than 50% may have a diameter between about 10 nm and about 300 nm, or more than 50% may have a diameter between about 50 nm and about 200 nm). The lipid vesicles may be coated or stabilized.

Lipid vesicles are used for drug delivery due to the properties of their lipid bilayer. Dissolved hydrophilic solutes cannot readily pass through the lipids. Hydrophobic chemicals can be dissolved into the lipid bilayer and, in this way, lipid vesicles can carry both hydrophobic molecules and hydrophilic molecules. To deliver THC and melatonin compounds as described herein to their sites in the body, the lipid bilayer can fuse with cellular membranes, thus delivering the lipid vesicle's contents. By preparing the lipid vesicles in a solution comprising THC and melatonin compounds as described herein, they are delivered to cells lining the digestive system.

Lipid vesicles can also be designed to deliver the compounds as described herein in other ways. As the pH is neutral within the lipid vesicles (protons can pass through their lipid bilayers), the compound may be neutralized, enabling it to freely pass through the lipid bilayer. These lipid vesicles work to deliver a compound as described herein by diffusion rather than by direct fusion. Another strategy for drug delivery is to target endocytosis events. Lipid vesicles can be made in a particular size range that makes them viable targets for natural macro-phage phagocytosis. These lipid vesicles may be digested while in the cell's phagosome, thus releasing the THC compound and/or the melatonin compound as described herein. Lipid vesicles can also be decorated with opsonins and ligands to activate endocytosis in other cell types.

Lipid vesicles may be prepared using conventional processes as known in the art. Lipid vesicles can be prepared by dissolving lipids in a solvent in a container. The container may have a volume ten times greater than the volume of the expected suspension of lipid vesicles. Using a rotary evaporator, the solvent is removed at about 40° C. under negative pressure. The solvent normally is removed within about 5 minutes to 2 hours, depending on the desired volume of the lipid vesicles. The composition can be dried further in a desiccator under vacuum. Dried lipids can be hydrated at about 25 mM-50 mM phospholipid in sterile, pyrogen free water by shaking until the lipid film is substantially resuspended. The lipid vesicles in aqueous solution can then be separated into aliquots, each placed in a vial, lyophilized, and sealed under vacuum.

As micelles and lipid vesicles contact aqueous solutions on both sides of their phospholipid bilayer, their amphiphilic phospholipids have their hydrophobic tails facing each other and their hydrophilic polar heads contacting water. The lipids may be selected from sterol lipids, fatty acids, fatty alcohols, glycerolipids (e.g., monoglycerides, diglycerides, and triglycerides), phospholipids, glycerophospholipids, sphingolipids, prenol lipids, saccharolipids, polyketides, or any combination thereof. The lipid can be a polyunsaturated fatty acid or alcohol. The terms polyunsaturated fatty acid and polyunsaturated fatty alcohol means a fatty acid or an alcohol, respectively, with two or more carbon-carbon double bonds in its hydro-carbon chain. The lipid can also be a highly unsaturated fatty acid or alcohol. The terms highly polyunsaturated fatty acid and highly polyunsaturated fatty alcohol means a fatty acid or an alcohol, respectively. having at least 18 carbon atoms and at least 3 double bonds. The lipid or phospholipid may be conjugated to polyethylene glycol (PEG). The lipid portion of the PEG conjugate may be diacylglycerol, dialkylamine, dialkylglycerol, phosphatidic acid, or phosphatidylethanolamine.

Specific examples of lipids include, but are not limited to, 1,3-propanediol dicaprylate/dicaprate, 10-undecenoic acid, 1-dotriacontanol, 1-heptacosanol, 1-nonacosanol, 2-ethyl hexanol, androstanes, arachidic acid, arachidonic acid, arachidyl alcohol, behenic acid, behenyl alcohol, capric acid, capric alcohol, capryl alcohol, caprylic acid, caprylic/capric acid ester of a saturated fatty alcohol C₁₂-C₁₈, caprylic/capric triglyceride, caprylic/capric triglyceride, ceramide phosphorylcholine, ceramide phosphorylethanolamine, ceramide phosphoryl-glycerol, ceroplastic acid, cerotic acid, cerotic acid, ceryl alcohol, cetearyl alcohol, cetyl alcohol, cholanes, cholestanes, cholesterol, cis-11-eicosenoic acid, cis-11-octadecenoic acid, cis-13-docosenoic acid, docosahexaenoic acid, eicosapentaenoic acid, eicosenoic acid, elaidic acid, elaidolinolenyl alcohol, elaidolinoleyl alcohol, elaidyl alcohol, erucic acid, erucyl alcohol, estranes, distearate, geddic acid, geddyl alcohol, glycerol tricaprylate/caprate, glycerol tricaprylate/caprate, glyceryl monocaprylate, glyceryl triacetate, glyceryl tricaprylate, glyceryl tricaprylate/caprate/laurate, glyceryl tricaprylate/tricaprate, glyceryl tripalmitate, henatriacon-tylic acid, heneicosyl alcohol, heneicosylic acid, heptacosylic acid, heptadecanoic acid, heptadecyl alcohol, hexatriacontylic acid, isostearic acid, isostearyl alcohol, lacceroic acid, laurie acid, lauryl alcohol, lignoceric acid, lignoceryl alcohol, linoelaidic acid, linoleic acid, linolenyl alcohol, linoleyl alcohol, margaric acid, melissic acid, melissyl alcohol, montanic acid, montanyl alcohol, myricyl alcohol, myristic acid, myristoleic acid, myristyl alcohol, neodecanoic acid, neoheptanoic acid, neononanoic acid, nervonic, nonacosylic acid, nonadecyl alcohol, nonadecylic acid, nonadecylic acid, oleic acid, oleyl alcohol, palmitic acid, palmitoleic acid, palmitoleyl alcohol, pelargonic acid, pelargonic alcohol, pentacosylic acid, pentadecyl alcohol, pentadecylic acid, phosphatidic acid, phosphatidylcholine, phosphatidylethanolamine, phosphatidylglycerol, phosphatidylserine, polyglyceryl-6-distearate, pregnanes, propylene glycol dicaprate, propylene glycol dicaprylocaprate, propylene glycol dicaprylocaprate, psyllic acid, recinoleaic acid, recinoleyl alcohol, sapienic acid, stearic acid, stearidonic, stearyl alcohol, tricosylic acid, tridecyl alcohol, tridecylic acid, undecyl alcohol, undecylenic acid, undecylic acid, alpha-linolenic acid, gamma-linolenic acid, or any combination thereof.

Specific examples of phospholipids include, but are not limited to, phosphatidic acid, phosphatidylcholine, phosphatidylethanolamine, phosphatidylglycerol, phosphatidylserine, lysophosphatidic acid, lysophosphatidylcholine, lysophosphatidylethanolamine, lysophosphatidylglycerol, lysophosphatidylserine, sphingomyelin, dimyristoyl-phosphatidylglycerol, dimyristoylphosphatidylcholine, dimyristoylphosphatidylethanolamine, dimyristoylphosphatidylserine, dioleoylphosphatidylglycerol, dioleoylphosphatidylcholine, dioleoylphosphatidylethanolamine, dioleoylphosphatidylserine, dipalmitoylphosphatidylgly-cerol, dipalmitoylphosphatidylcholine, dipalmitoylphosphatidylethanolamine, distearoylphos-phatidylglycerol, distearoylphosphatidylcholine, distearoylphosphatidylethanolamine, palmi-toyloleoylphosphatidylcholine, stearoylpalmitoylphosphatidylcholine, or any combination thereof.

### Combination Therapy

THC and melatonin compounds as described herein may be administered in combination with one or more other treatments or therapies. For example, provided herein are methods for combination therapy in which at least one chemotherapeutic agent known to modulate at least a signaling pathway other than those associated with cannabinoid and melatonin receptors, or even components of cannabinoid or melatonin signaling pathways is used in combination therapy with the THC and melatonin compounds as described herein, or a pharmaceutically-acceptable form of either compound or both compounds (e.g., one or more pharmaceutically-acceptable salts of a THC compound and/or a melatonin compound as described herein, one or more pharmaceutically-acceptable solvates of the THC compound and/or the melatonin compound such as hydrates of the THC compound and/or the melatonin compound, one or more pharmaceutically-acceptable isomers of the THC compound and/or the melatonin compound, one or more pharmaceutically-acceptable prodrugs of the THC compound and/or the melatonin compound). Systems and protocols of the invention include, but are not limited to, use of a combination of THC and melatonin compounds as described herein (and optional polyphenolic compounds) together with at least one chemotherapeutic agent (including, but not limited to, small molecule drugs, antibodies, other biologics) to provide at least a therapeutic, a prophylactic, a preventive, a palliative, or another therapeutic effect.

The terms “combination therapy” and “administered in combination” are used interchangeably. They encompass simultaneous, synchronous, nonsynchronous, or sequential administration of at least a THC compound and a melatonin compound as described herein, optional polyphenolic compounds (e.g., curcumin and rutin, compounds related thereto), optional Vitamin E-related compound, and at least one chemotherapeutic agent to a subject in need of treatment. The THC and melatonin compounds as described herein and the at least one chemotherapeutic agent are therapeutically active compounds. Polyphenolic and Vitamin E-related compounds may also be considered therapeutically active compounds, but they are not necessarily functioning effectively in that capacity. In certain embodiments, combination therapy comprises administering therapeutically active compounds in at least two, three, or four separate compositions; administering them at two, three, or four different times in two, three, or four separate compositions; or administering them at two, three, or four different times each separated by at least one or two hours in two, three, or four separate compositions to a subject in need of treatment.

By “in combination with,” it is not intended to imply that the at least one chemotherapeutic agent must be administered concurrently and/or formulated for delivery together with THC and melatonin compounds as described herein, although these methods of delivery are within the scope of combination therapy. THC and melatonin compounds as described herein can be administered concurrently with, prior to (e.g., 5 minutes, 15 minutes, 30 minutes, 45 minutes, 1 hour, 2 hours, 4 hours, 6 hours, or 12 hours before), or subsequent to (e.g., 5 minutes, 15 minutes, 30 minutes, 45 minutes, 1 hour, 2 hours, 4 hours, 6 hours, or 12 hours after), other treatments using one or more additional chemotherapeutic agents. In general, each chemotherapeutic agent will be administered at a daily dosage and on a dosing schedule determined for that particular chemotherapeutic agent before treatment. At least one chemotherapeutic agent can be administered with the THC and melatonin compounds as described herein in the same composition or separately in a different composition.

In general, it is expected that any additional chemotherapeutic agents employed in combination with protocols as described herein are to be utilized at levels that do not exceed the levels at which they are utilized by themselves. In some embodiments, the levels utilized in combination will be lower than those utilized by themselves.

In another embodiment, compositions for treating disease may comprise therapeutically effective amounts of the THC and melatonin compounds as described herein or a pharmaceutically-acceptable form (e.g., a salt, a solvate, a hydrate, a salt of a solvate or a hydrate) thereof, in combination with a therapeutically effective amount of at least one chemotherapeutic agent used to treat dementia or Alzheimer's disease (e.g., aducanumab, amantadine, bepranemab, brexpiprazole, bryostatin, colostrinin, donanemab, donepezil, Ginko biloba extract EGb 761, galantamine, gantesnerumab, gosuranemab, lanabecestat, lecanemab, leucomethylthioninium, levetiracetam, memantine, methylphenidate, neflamapimod, prasinezumab, rivastigmine, semorinemab, solanezumab, tilavonemab, tramiprosate, tricaprilin, zagotenemab, or any combination thereof). As an alternative chemotherapeutic agent, any of the chemical and biological compounds for treating dementia, Alzheimer's disease, depression, an anxiety disorder, panic disorder, or a sleep disorder that are known in the art for their off-label use or were approved by medical regulatory authorities for treatment. The chemotherapeutic agent may or may not be used in combination with the THC and melatonin compounds as described herein.

At least one chemotherapeutic agent can be combined with the two or more compounds as described herein (e.g., THC compounds; melatonin compounds; optional polyphenolic compounds such as curcumin and rutin, optional Vitamin E, and compounds related thereto). A chemotherapeutic agent may be selected from one or more of the following: antidepressants, antipsychotics, mood stabilizers, selective serotonin reuptake inhibitors, serotonin/norepinephrine reuptake inhibitors, and anxiolytics. For example, the chemotherapeutic agent may be selected from, without limitation, selective serotonin reuptake inhibitors (e.g., citalopram, escitalopram, fluvoxamine, sertraline, vortioxetine); serotonin/norepinephrine reuptake inhibitors (e.g., desvenlafaxine, duloxetine, levomilnacipran, venlafaxine); antidepressants (e.g., mirtazapine, trazodone); mood stabilizers (e.g., carbamazepine, gabapentin, lamotrigine, oxcarbazepine topiramate, valproic acid); antipsychotics (e.g., aripiprazole, haloperidol, olanzapine, perphenazine, quetiapine, risperidone, ziprasidone); anxiolytics (e.g., buspirone, gepirone, ipsapirone, tandospirone); and orexin receptor antagonists (e.g., lemborexant, suvorexant). The chemotherapeutic agent may or may not be used in combination with the THC and melatonin compounds as described herein.

The inventors show below that certain symptoms of neurologic disease are improved by administering THC and melatonin at an intermediate dosage.

### NON-LIMITING EXAMPLES

According to npiTEST.net, its Neuropsychiatric Inventory (NPI) is the most widely used outcome measure in clinical trials of drugs to treat dementia. Patient behaviors associated with dementia and Alzheimer's disease may be assessed by the NPI and similar tests, and changes may be quantitated using their rating scales. Clinical trials have adopted the NPI to evaluate the effectiveness of antidementia drug treatments by their ability to improve symptoms of Alzheimer's disease in the following behavioral domains: ten neuropsychiatric symptoms (i.e., delusions, hallucinations, agitation/aggression, depression, anxiety, elation/euphoria, apathy, disinhibition, irritability, and aberrant motor behavior) and two neurovegetative symptoms (i.e., sleep disorders and appetite/eating disorders).

Cummings describes the NPI as a comprehensive assessment of psychopathology in patients with dementia. A neuropsychiatric disorder may be diagnosed from its characteristic symptoms, such as changes in behavior, as well as distinctive NPI profiles. The reliability, reproducibility, and validity of the NPI for assessing behavioral symptoms indicative of dementia and Alzheimer's disease have been independently confirmed. There is no cut-off score indicating an abnormal NPI.

The Neuropsychiatric Inventory-Questionnaire (NPI-Q) was adapted from the Neuropsychiatric Inventory and provides a brief assessment of a patient by interviewing an informant caring for the patient and familiar with his behavior over the past month. The NPI-Q contains questions relating to Alzheimer's disease symptoms in the 12 behavioral domains listed above. The frequency and severity of each behavior are rated by the patient's caregiver. The caregiver also rates her own distress caused by the patient's behavior. Survey responses were tallied, and scores were calculated: patient total score, caregiver distress score, and scores for each of the 12 problematic behaviors. The NPI is a reliable, reproducible, and validated assessment instrument for studying the neuropsychiatric manifestations and symptoms of dementia syndromes, diagnosing and distinguishing among dementia syndromes, and evaluating the effectiveness of investigative treatments on the natural history of Alzheimer's disease. It is sensitive to treatment effects and has demonstrated in clinical trials that cholinergic agents can improve behavioral symptoms of Alzheimer's disease patients.

Initial results were obtained from a randomized, triple-blinded, crossover clinical trial (NCT04749563) having ascending daily dosages and a placebo control. This phase 1 study evaluated safety and tolerability in three dose escalation cohorts in the same group of 12 patients with dementia due to Alzheimer's disease. Given the vulnerability of the study population, a safety cohort (two patients active, one patient placebo) started every dose one day ahead of the other patients for early detection of adverse events. Safety and tolerability of the three dosages were chosen as the primary outcome measure. Changes in NPI scores from baseline were chosen as secondary outcome measures. A change in the NPI's Patient Total or Caregiver Distress score was used as a sensitive measure of patient outcome in clinical trials of antidementia drugs (e.g., acetylcholinesterase inhibitors).

The caregiver is read each question exactly as written. If the caregiver fails to comprehend a question, it is repeated or asked in alternate terms. After reading the screening question, the caregiver is asked if the behavior described is present. If the answer is “No” the clinician proceeds to the next section and reads the next screening question. But if the answer is “Yes” then additional questions are read and “Yes” or “No” responses are obtained: the caregiver is asked to rate the frequency (i.e., occasionally, often, frequently, or very frequently) and severity (i.e., mild, moderate, or severe) of the patient's most abnormal behavior. After the frequency and severity of each behavior is determined, the caregiver is asked to rate her own distress associated with that behavior (i.e., no distress, minimal, mild, moderate, moderate severe, or very severe or extreme) before the clinician proceeds to the next behavioral domain.

### Frequency of the Patient's Behavior

1=Occasionally (less than once per week)
2=Often (about once per week)
3=Frequently (several times per week but not daily)
4=Very Frequently (daily or essentially continuously present)

### Severity of the Patient's Behavior

1=Mild (noticeable, but not a significant change in behavior)
2=Moderate (significant, but not a dramatic change in behavior)
3=Severe (very marked or prominent, a dramatic change)

### Caregiver Distress

(Response to “How emotionally distressing do you find this behavior?”)
0=No Distress
1=Minimal
2=Mild
3=Moderate
4=Moderate Severe
5=Very Severe or Extreme

Frequency, Severity, and Caregiver Distress were scored according to the above rating scales. NPI score was calculated as the product of Frequency and Severity for each symptom (i.e., delusions, hallucinations, agitation/aggression, depression, anxiety, elation/euphoria, apathy, disinhibition, irritability, aberrant motor behavior, sleep disorders, and appetite/eating disorders). The Patient Total score was calculated as the sum of the products Frequency x Severity for the 12 behavioral domains.

Patients were enterally administered one, two, or three capsules daily. Each 1 ml capsule consists of 2.5 mg THC, 1.5 mg melatonin, 0.5 mg curcumin, 0.5 mg rutin, 10 mg ascorbic acid, 0.025 ml Vitamin E TPGS (about 18.75 mg d-alpha-tocopherol), 0.01 ml Tween 80 (polysorbate 80), 0.12 ml ethanol, 0.2 ml honey, and water up to total volume of 1 ml. A cohort of 12 patients having Alzheimer's disease was treated with the Low 1× daily dosage for two weeks, followed by a washout period of four days. The same cohort was then given the Medium 2× daily dosage for two weeks, followed by another washout period of four days. Finally, the cohort was given the High 3× daily dosage for two weeks. Out of the cohort of 12 patients, two received placebo.
I. Low was one dose of 2.5 mg THC and 1.5 mg melatonin daily for 2 weeks
II. Medium was one dose of 5 mg THC and 3 mg melatonin daily for 2 weeks
III. High was one dose of 7.5 mg THC and 4.5 mg melatonin daily for 2 weeks

A patient's neuropsychiatric symptom was considered improved if there was a statistically significant difference (decrease) in the change from baseline of mean NPI score for the corresponding behavioral domain. The secondary outcome criterion was considered satisfied if there was a statistically significant decrease in either mean Patient Total change score and/or mean Caregiver Distress change score. NPI score for each behavioral domain was obtained in interviews on Day 1 (before treatment or baseline), Day 10 (during treatment), and Day 15 (end of treatment). The change scores after one week and two weeks of treatment were calculated for each patient, then the mean change score was calculated for the treatment group. A statistically significant difference in the mean NPI score (i.e., change from baseline) rejects the null hypothesis. A symptom is considered to have improved due to treatment if there was a significant decrease in mean NPI change score for the treated group; a significant increase in mean NPI change score for the treated group would have been considered worsening of the symptom.

Because the NPI score could have changed in either direction due to treatment (i.e., a symptom improved or worsened), a two-tailed test was used. The two-tailed test compared change scores “Day 1 vs. Day 10” and “Day 1 vs. Day 15” with the null hypothesis being No Difference Due to Treatment. At a significance level of 0.05 (5%), the two-tailed test allots half (i.e., 0.025 or 2.5%) to testing significance in one direction (symptom improved) and half to testing significance in the other direction (symptom worsened). A p-value <0.025 rejects the null hypothesis. The p-values for Low daily dosage treatment are shown in Table I, the p-values for Medium daily dosage treatment are shown in Table II, and the p-values for High daily dosage treatment are shown in Table III.

At the Low daily dosage of 2.5 mg THC and 1.5 mg melatonin, the null hypotheses were rejected for two out of 12 behavioral domains after one week of treatment. There were significant improvements in depression and anxiety. The null hypothesis was also rejected after two weeks of Low daily dosage treatment for depression. The change in mean Patient Total score from the baseline score (i.e., Patient Total score) also improved after one week of Low daily dosage treatment and stayed improved at the end of treatment. The same pattern was seen for the change in mean Caretaker Distress score from the baseline score (i.e., Caretake Distress score).

At the Medium daily dosage of 5.0 mg THC and 3.0 mg melatonin, the null hypotheses were rejected for three out of 12 behavioral domains after one week of treatment. The null hypothesis was also rejected after two weeks of Medium daily dosage treatment for depression. Patient Total score improved after one week of Medium daily dosage treatment and stayed improved at the end of treatment. The same pattern of improvement was seen for Caretaker Distress score. In general, treatment with the Medium daily dosage benefited Alzheimer's disease more than the Low daily dosage.

At the High daily dosage of 7.5 mg THC and 4.5 mg melatonin, the null hypothesis was not rejected for any of the 12 behaviors after one week or two weeks of treatment. Surprisingly, increasing the amounts of THC and melatonin did not increase the benefit to Alzheimer's disease patients seen at the two lower doses. Patient Total score only improved after two weeks of High daily dosage treatment. Caretaker Distress score never improved.

Table IV is a summary of the changes from baselines of mean Patient Total score and mean Caretaker Distress score after one week (Day 1 minus Day 10) and two weeks (Day 1 minus Day 15) of each treatment. For convenience, the p-values from Table I to III are repeated in Table IV to compare head-to-head Low, Medium, and High daily dosage treatments. As noted above, there were impressive improvements in Patient Total and Caretaker Distress scores due to Low and Medium daily dosage treatments. Patient Total score improvement was seen as quickly as one week of treatment and continued after the second week of treatment. Similarly, Caregiver Distress score was improved due to Low and Medium daily dosage treatments. But at the High daily dosage, Patient Total score did not improve until after two weeks of treatment and Caretaker Distress score never improved.

We conclude that the most dramatic effects on patient outcome were obtained by treatment at daily dosages lower than the maximum level reached in our trial. The improvements were rapid and lasting. It should also be noted that the changes in mean scores for the treated group never went in the other direction (i.e., worsening). Thus, treatment, even at the High daily dosage, did not worsen any of the 12 neuropsychiatric symptoms assessed in our trial.

Howard et al. (Int J Geriatric Psychiatry, 2011 August, 26: 812-817) published a minimum clinically important difference for Patient Total score of 8 (i.e., 0.4 of standard deviation in the change from baseline). In Table IV, the statistically significant difference in change scores satisfies Howard's criterion.

npiTEST.net stated the clinical meaningfulness of a drug-placebo difference will vary according to baseline severity of the behavioral change and the domain of behavior affects. “As a general rule, a decrease in 4 points or a 30% reduction in baseline score would be regarded as clinically meaningful” according to npiTEST.net. “However, a smaller change in some symptoms such as agitation might be viewed as important by caregivers or might indicate an important drug effect. This must be considered on a case-by-case basis and is optimally pre-specified in the analysis.” Here, no statistically significant difference between treated and placebo groups was calculated. The small number of patients and their large variance in NPI scores may have resulted in changes due to treatment that did not rise to statistical significance. Are there patient characteristics that could be used to predict who benefits from treatment?Recruiting patients using such criteria might reduce variance in their baseline and change scores. Recruiting more patients to increase sample size (e.g., both placebo and treatment groups) should decrease the width of confidence intervals. Therefore, recruiting patients having late-onset Alzheimer's disease, who are younger, lack serious comorbidities, are otherwise healthy and well, or any combination thereof; or whose symptoms, cognitive deficits, functional impairments, or any combination thereof are mild, as well as enlarging both treatment and placebo groups, might enable discriminating a minimal important difference.

TABLE I
Improvements Due to Low Dose Treatment of
2.5 mg THC and 1.5 mg Melatonin (p < 0.025)

| Patient | Day 1 vs. | Day 1 vs. | Caregiver | Day 1 vs. | Day 1 vs. |
|---|---|---|---|---|---|
| Behavioral Domains | Day 10 | Day 15 | Behavioral Domains | Day 10 | Day 15 |
| Delusions | 0.17 | 0.06 | Delusions | 0.22 | 0.05 |
| Hallucinations | 0.41 | 0.07 | Hallucinations | 0.56 | 0.14 |
| Agitation/Aggression | 0.06 | 0.05 | Agitation/Aggression | 0.04 | 0.02 |
| Depression | 0.02 | 0.01 | Depression | 0.03 | 0.07 |
| Anxiety | 0.02 | 0.09 | Anxiety | 0.01 | 0.02 |
| Elation/Euphoria | 0.34 | 0.34 | Elation/Euphoria | 0.34 | 0.34 |
| Apathy | 0.23 | 0.11 | Apathy | 0.31 | 0.08 |
| Disinhibition | 0.48 | 0.38 | Disinhibition | 0.49 | 0.64 |
| Irritability | 0.04 | 0.12 | Irritability | 0.10 | 0.44 |
| Aberrant Motor | 0.12 | 0.10 | Aberrant Motor | 0.09 | 0.43 |
| Behavior | | | Behavior | | |
| Sleep Disorders | 0.11 | 0.08 | Sleep Disorders | 0.15 | 0.09 |
| Appetite/Eating | 0.05 | 0.01 | Appetite/Eating | 0.03 | 0.07 |
| Disorders | | | Disorders | | |
| TOTAL | 0.004 | 0.01 | Distress | 0.01 | 0.01 |

TABLE II
Improvements Due to Medium Dose Treatment
of 5 mg THC and 3 mg Melatonin (p < 0.025)

| Patient | Day 1 vs. | Day 1 vs. | Caregiver | Day 1 vs. | Day 1 vs. |
|---|---|---|---|---|---|
| Behavioral Domains | Day 10 | Day 15 | Behavioral Domains | Day 10 | Day 15 |
| Delusions | 0.17 | 0.83 | Delusions | 0.18 | 0.50 |
| Hallucinations | 0.19 | 0.17 | Hallucinations | 0.19 | 0.19 |
| Agitation/Aggression | 0.24 | 0.06 | Agitation/Aggression | 0.10 | 0.04 |
| Depression | 0.01 | 0.01 | Depression | 0.05 | 0.01 |
| Anxiety | 0.01 | 0.27 | Anxiety | 0.05 | 0.05 |
| Elation/Euphoria | 0.34 | 0.34 | Elation/Euphoria | 0.34 | 0.34 |
| Apathy | 0.49 | 0.13 | Apathy | 0.05 | 0.03 |
| Disinhibition | 0.77 | 0.19 | Disinhibition | 0.56 | 0.28 |
| Irritability | 0.02 | 0.04 | Irritability | 0.03 | 0.05 |
| Aberrant Motor | 0.06 | 0.52 | Aberrant Motor | 0.11 | 0.28 |
| Behavior | | | Behavior | | |
| Sleep Disorders | 0.30 | 0.30 | Sleep Disorders | 0.56 | 0.56 |
| Appetite/Eating | 0.05 | 0.32 | Appetite/Eating | 0.07 | 0.18 |
| Disorders | | | Disorders | | |
| TOTAL | 0.003 | 0.01 | Distress | 0.01 | 0.01 |

TABLE III
Improvements Due to High Dose Treatment of
7.5 mg THC and 4.5 mg Melatonin (p < 0.025)

| Patient | Day 1 vs. | Day 1 vs. | Caregiver | Day 1 vs. | Day 1 vs. |
|---|---|---|---|---|---|
| Behavioral Domains | Day 10 | Day 15 | Behavioral Domains | Day 10 | Day 15 |
| Delusions | 0.19 | 0.15 | Delusions | 0.18 | 0.34 |
| Hallucinations | 0.17 | 0.17 | Hallucinations | 0.19 | 0.19 |
| Agitation/Aggression | 0.87 | 0.06 | Agitation/Aggression | 0.34 | 0.07 |
| Depression | 0.73 | 0.15 | Depression | 0.19 | 0.11 |
| Anxiety | 0.55 | 0.18 | Anxiety | 0.19 | 0.18 |
| Elation/Euphoria | 0.34 | 0.34 | Elation/Euphoria | 0.34 | 0.34 |
| Apathy | 0.59 | 0.22 | Apathy | 0.76 | 0.68 |
| Disinhibition | 0.34 | 0.34 | Disinhibition | 0.34 | 0.68 |
| Irritability | 0.90 | 0.22 | Irritability | 0.84 | 0.11 |
| Aberrant Motor | 0.68 | 0.73 | Aberrant Motor | 1.00 | 0.44 |
| Behavior | | | Behavior | | |
| Sleep Disorders | 0.39 | 0.06 | Sleep Disorders | 0.56 | 0.18 |
| Appetite/Eating | 0.05 | 0.17 | Appetite/Eating | 0.59 | 0.68 |
| Disorders | | | Disorders | | |
| TOTAL | 0.72 | 0.01 | Distress | 0.61 | 0.03 |

TABLE IV
Comparing Improved Patient Total and Caregiver Distress Scores

| Patient | One | Two | Caregiver | One | Two |
|---|---|---|---|---|---|
| Total | Week | Weeks | Distress | Week | Weeks |
| Low Dose | ↓15 | ↓17 | Low Dose | ↓8 | ↓7 |
| p-value | 0.004 | 0.01 | p-value | 0.01 | 0.01 |
| Medium Dose | ↓12 | ↓10 | Medium Dose | ↓7 | ↓7 |
| p-value | 0.003 | 0.01 | p-value | 0.01 | 0.01 |
| High Dose | ↓1 | ↓8 | High Dose | ↓1 | ↓3 |
| p-value | 0.72 | 0.01 | p-value | 0.61 | 0.03 |

TABLE V
Comparing Claimed Daily Dosage
to Examples of WO 2019/190608

| | Ex. 1 - | Ex. 9 - | Ex. 10 - | Claimed |
|---|---|---|---|---|
| Component | 1 ml | 1 ml | 1 ml | Daily Dosage |
| THC | 1.5 mg | 1.5 mg | 7.5 mg | 2 mg-7 mg |
| CBD | none | none | 7.5 mg-150 mg | none |
| Melatonin | 1.5 mg | 1.5 mg | 4.5 mg | 1.0 mg-4.4 mg |
| Curcumin | 1.0 mg | 1.0 mg | 1.5 mg | 0.3 mg-1.5 mg |
| Rutin | none | 1.1 mg | 1.5 mg | 0.3 mg-1.5 mg |

### REFERENCES

1. CANEVELLI et al. “Behavioral and psychological subsyndromes in Alzheimer's disease using the Neuropsychiatric Inventory” Intl J Geriat Psychiatry, 2013 August, 28: 795-803.
2. CONNORS et al. “The stability of neuropsychiatric subsyndromes in Alzheimer's disease” Alzheimers Dement, 2018 July, 14: 880-888.
3. CUMMINGS et al. “The Neuropsychiatric Inventory: Comprehensive assessment of psychopathology in dementia” Neurology, 1994 December, 44: 2308-2314.
4. CUMMINGS “The Neuropsychiatric Inventory: Assessing psychopathology in dementia patients” Neurology, 1997 May, 48: S10-S16.
5. CUMMINGS & McPHERSON “Neuropsychiatric assessment of Alzheimer's disease and related dementias” Aging, 2001 June, 13: 240-246.
6. CUMMINGS & ZHONG “Trial design innovations: Clinical trials for treatment of neuropsychiatric symptoms in Alzheimer's disease” Clin Pharmacol Ther, 2015 November, 98: 483-485.
7. CUMMINGS “The Neuropsychiatric Inventory: Development and applications” J Geriatr Psychiatry Neurol, 2020 March, 33: 73-84.
8. EIKELBOOM et al. “Neuropsychiatric and cognitive symptoms across the Alzheimer disease clinical spectrum: Cross-sectional and longitudinal associations” Neurology, 2021 August, 97: e1276-e1287, doi: 10.1212/WNL.0000000000012598.
9. FRISONI et al. “Behavioral syndromes in Alzheimer's disease: Description and correlates” Dement Geriatr Cogn Disord, 1999 March, 10: 130-138.
10. KAUFER et al. “Assessing the impact of neuropsychiatric symptoms in Alzheimer's disease: The Neuropsychiatric Inventory Caregiver Distress Scale” J Am Geriatr Soc, 1998 February, 46: 210-215.
11. KAUFER et al. “Validation of the NPI-Q, a brief clinical form of the Neuropsychiatric Inventory” J Neuropsychiatry Clin Neurosci, 2000 Spring, 12: 233-239.
12. LAI “The merits and problems of Neuropsychiatric Inventory and an assessment tool in people with dementia and other neurological disorders” Clin Interv Aging, 2014 July, 9: 1051-1061.
13. LANCTOT et al. “Neuropsychiatric signs and symptoms of Alzheimer's disease: New treatment paradigms” Alzheimers Dement, 2017 August, 3: 440-449.
14. de MEDEIROS et al. “The Neuropsychiatric Inventory-Clinician rating scale (NPI-C): Reliability and validity of a revised assessment of neuropsychiatric symptoms in dementia” Int Psychgeriatr, 2010 September, 22: 984-994.
15. SAARI et al. “Psychometric properties of the Neuropsychiatric Inventory” J Alzheimer Dis, 2020 August, 86: 1485-1499, doi: 10.3233/JAD-200739.

The publications cited above describe various versions of the NPI, their validation, and their use in assessing symptoms of patients having Alzheimer's disease. All cited publications and U.S. Patent Application 63/245,799 are incorporated by reference herein.

Table V compares the daily dosage of claims 1, 18 and 48 below to compositions from examples of the inventors' WO 2019/190608. The claimed daily dosage includes the Low and Medium daily dosage, but not the High daily dosage. As a consequence, the claimed range for THC excludes the disclosed amounts of THC in WO 2019/190608. In other words, “from about 2 mg (or more) to about 7 mg (or less)” of THC excludes about 1.5 mg and about 7.5 mg THC. Similarly, “from about 1.6 mg (or more)” of melatonin excludes about 1.5 mg melatonin and “to about 4.4 mg (or less)” of melatonin excludes about 4.5 mg melatonin.

Aspects of the inventors' discovery and their invention, their conjecture on a principle of operation, and features they not yet claimed are described above. Although specific embodiments applying aspects of the invention are contemplated, successful implementation of every contemplated embodiment is not required to benefit from the inventors' discovery. Similarly, their conjecture about a principle of operation, whether or not borne out, does not negate in any way the usefulness and practice of the invention as claimed.

While their description may refer to certain alternative elements or materials, design choices or ornamental designs, functional limitations, goals or objectives, means or steps, optional arrangements or configurations, preferences, relative or subjective terms, sensory perceptions (e.g., colors, flavors, odors, sounds, textures), shapes or sizes, or the like, it would be understood that the inventors' discovery and invention may be adapted or modified by following, in general, their operating principles, and including such departures from the description that are customary practice or known in the art to which the invention pertains, and as may be applied to the central features hereinbefore set forth, and fall within the scope of the invention and of the limits of the claims appended hereto or presented later. The claims, therefore, should not be limited in their scope of protection to specific embodiments and examples described in the specification.

1. A daily dosage for treating one or more behavioral and psychological symptoms of a human patient in need of such treatment, characterized as the daily dosage comprising:
(i) an effective amount of a tetrahydrocannabinol (THC) compound, wherein its amount is from about 2.0 mg to about 7.0 mg,
(ii) an effective amount of a melatonin compound, wherein its amount is from about 1.0 mg to about 4.4 mg.

2. The daily dosage of claim 1, wherein the amount of the THC compound is from about 2.25 mg to about 6.75 mg.

3. The daily dosage of claim 1, wherein the amount of the THC compound is from about 2.5 mg to about 6.5 mg.

4. The daily dosage of claim 1, wherein the THC compound is selected from the group consisting of dronabinol, nabilone, trans-delta-8-THC (Δ8-THC), trans-delta-9-THC (Δ9-THC), Δ9-THC stereoisomers, Δ9-THC carboxylic acid (THCA), Δ9-THC-4-oic acid (THCA-A), Δ9-THC-2-oic acid (THCA-B), 11-hydroxy-Δ9-THC (11-OH-THC), 11-nor-9-carboxy-Δ9-THC (11-COOH-THC), 7,8,9,10-tetrahydro-6,6,9-trimethyl-3-pentyl-6H-dibenzo[b,d]pyran-1-ol (Δ6a, 10a-THC), (9R,10aR)-8,9,10,10a-tetrahydro-6,6,9-trimethyl-3-pentyl-6H-dibenzo[b,d]pyran-1-ol (Δ6a, 7-THC), (6aR,9R,10aR)-6a,9,10,10a-tetrahydro-6,6,9-trimethyl-3-pentyl-6H-dibenzo[b,d]pyran-1-ol (Δ7-THC), 6a,7,8,9-tetrahydro-6,6,9-trimethyl-3-pentyl-6H-dibenzo[b,d]pyran-1-ol (Δ10-THC), and (6aR,10aR)-6a,7,8,9,10,10a-hexahydro-6,6-dimethyl-9-methylene-3-pentyl-6H-dibenzo[b,d]pyran-1-ol (Δ9, 11-THC), anandamide, 2-arachidonoyl-glycerol, 2-arachidonoyl-glyceryl ether, and all combinations thereof.

5. The daily dosage of claim 1, wherein the amount of the melatonin compound is from about 1.25 mg to about 4.2 mg.

6. The daily dosage of claim 1, wherein the amount of the melatonin compound is from about 1.5 mg to about 4.0 mg.

7. The daily dosage of claim 1, wherein the melatonin compound is selected from the group consisting of circadin, N-acetyl-5-methoxytryptamine, agomelatine, ramelteon, tasimelteon, 5-methoxytryptamine, N-acetyl-serotonin, and all combinations thereof.

8. The daily dosage of claim 1 further comprising a curcumin related compound in an amount from about 0.3 mg to about 1.5 mg.

9. The daily dosage of claim 1 further comprising a curcumin related compound in an amount from about 0.6 mg to about 1.2 mg.

10. The daily dosage of claim 8, wherein the curcumin related compound is selected from the group consisting of curcumin, turmeric, curcuminoids other than curcumin, structural derivatives of curcumin, analogs of curcumin that are enhanced for at least better absorption, bioavailability, stability, or taste, and all combinations thereof.

11. The daily dosage of claim 1 further comprising a rutin related compound in an amount from about 0.3 mg to about 1.5 mg.

12. The daily dosage of claim 1 further comprising a rutin related compound in an amount from about 0.6 mg to about 1.2 mg.

13. The daily dosage of claim 11, wherein the rutin related compound is selected from the group consisting of rutin, quercetin glycosides other than rutin, quercetrin, rutinosides other than rutin, hesperidin, structural derivatives of rutin, analogs of rutin that enhanced for at least better absorption, bioavailability, stability, or taste, and all combinations thereof.

14. The daily dosage of claim 1 further comprising two or more polyphenolic compounds selected from the group consisting of turmeric, curcuminoids, curcumin, quercetin glycosides, rutin, and rutinosides.

15. The daily dosage of claim 1, which is effective for treating at least one of dementia, Alzheimer's disease, major neurocognitive disorder, mild cognitive impairment, mild neurocognitive disorder, dementia-related psychosis, depression, major depressive disorder, an anxiety disorder, panic disorder, a sleep disorder, or an eating disorder.

16. The daily dosage of claim 1 in a liquid carrier, wherein at least some THC is formulated in alcoholic water, an oil, an emulsion comprising surfactants, or a phospholipid bilayer to enhance stability and solubility of THC.

17. The daily dosage of claim 16, wherein the surfactants are selected from the group consisting of polysorbate 80 and polyethylene glycol 1000 ester of Vitamin E succinate (TPGS).

18. The daily dosage of claim 1 comprising less than about 2% (wt/vol) cannabidiol (CBD).

19. A method of treating one or more behavioral and psychological symptoms, the method comprising administering a medicament daily to a human patient in need of such treatment, characterized as the medicament comprising:
(i) an effective amount of a tetrahydrocannabinol (THC) compound, wherein its amount is from about 2.0 mg to about 7.0 mg,
(ii) an effective amount of a melatonin compound, wherein its amount is from about 1.0 mg to about 4.4 mg

20.-49. (canceled)

50. A kit of parts or medical device, comprising:
(a) a medicament comprising one or more unit doses, each unit dose having:
(i) an effective amount of a tetrahydrocannabinol (THC) compound, wherein its amount is from about 2.0 mg to about 7.0 mg,
(ii) an effective amount of a melatonin compound, wherein its amount is from about 1.6 mg to about 4.4 mg;
(b) a package or an optional dispenser for a plurality of unit doses;
(c) a package insert comprising the medicament's prescription information;
(d) an expiration date of the medicament;
(e) a seal that at least resists tampering with the medicament.

51. (canceled)
